(19) United States
(12) Patent Application Publication
(10) Pub. No.: US 2026/0199706 A1
(43) Pub. Date: Jul. 16, 2026

(54) RADIATION DOSIMETRY APPARATUS AND RELATED METHODS

(71) Applicant: DALHOUSIE UNIVERSITY, Halifax (CA)

(72) Inventors: James Leonard ROBAR, Halifax (CA); Thalat Theresa MONAJEMI, Halifax (CA); Nicholas James LYNCH, Halifax (CA)

(21) Appl. No.: 19/134,645

(22) PCT Filed: Nov. 29, 2023
(86) PCT No.: PCT/CA2023/051598

§ 371 Date: May 30, 2025

Related U.S. Application Data
(60) Provisional application No. 63/429,865, filed on Dec. 2, 2022.

(51) Int. Cl. A61N 5/10; G01T 1/02; G01T 1/08; G01T 7/00; G06T 7/00; G06T 7/70

(52) U.S. Cl. CPC A61N 5/1071; G01T 1/023; G01T 1/08; G06T 7/70; G06T 7/97; A61N 2005/1059; G01T 7/005; G06T 2207/10012

(57) ABSTRACT

A radiation dosimetry apparatus comprises a body that supports scintillator elements that emit light when exposed to radiation. The body may be shaped to conform to contours of a portion of a patient's anatomy. Radiation intensity and dose may be determined by processing images obtained by one or more cameras to measure intensity of light emitted by different ones of the scintillator elements. The scintillator elements may be distributed non-uniformly based on a patient-specific radiation treatment plan. Scintillator elements may be arranged to provide increased spatial resolution and/or dose resolution in higher dose regions and/or regions corresponding to organs at risk. Radiation dosimeter apparatuses may be fabricated by additive manufacturing. Design and fabrication of the radiation dosimetry apparatuses may be integrated into workflows in clinical radiation treatment settings. Methods and systems for fabricating, calibrating, and using the radiation dosimetry apparatuses are described.

### CROSS-REFERENCE TO RELATED APPLICATION

This application claims priority from U.S. application No. 63/429,865 filed 2 Dec. 2022 and entitled RADIATION DOSIMETRY APPARATUS AND RELATED METHODS which is hereby incorporated herein by reference for all purposes. For purposes of the United States of America, this application claims the benefit under 35 U.S.C. § 119 of U.S. application No. 63/429,865 filed 2 Dec. 2022 and entitled RADIATION DOSIMETRY APPARATUS AND RELATED METHODS which is hereby incorporated herein by reference for all purposes.

### FIELD

This invention relates to the field of radiation treatment. More particularly the invention relates to apparatus for real time monitoring of radiation dose to a patient, methods for making the apparatus and dosimetry methods that apply the apparatus.

### BACKGROUND

Radiation therapy (RT) has been found to be extremely valuable for treating a range of conditions including certain cancers. While radiation delivered to a specific target can be very beneficial (for example, life extending or lifesaving) the amount of radiation that is delivered to a patient must be carefully controlled. Delivering an excessive radiation dose to a patient can be harmful or even fatal. Also, it is not possible, in general to deliver radiation to a target volume in or on a patient's body without also delivering radiation to other parts of the patient's body because radiation penetrates tissues. It is desirable to apply radiation in a way that delivers a prescribed radiation dose to a target volume while minimizing radiation dose to tissues outside of the target volume. It is especially important to minimize radiation dose to sensitive and/or critical tissues (“organs at risk” or “OAR”s) that are outside of the target volume.

RT is used in the treatment and management of cancer for approximately 50-60% of all cancer patients. RT most commonly involves external beam radiation therapy, whereby an x-ray or electron beam is generated by a treatment unit, most commonly a linear accelerator. The linear accelerator is typically controlled to direct multiple beams, or arcs of beams, all of which converge upon a target in a patient (for example, the target may be the volume of a tumor). To minimize the toxicity of the technique, radiation beams are typically shaped by a collimator (e.g. a multi-leaf collimator or MLC) to match the projection of the target volume or to create an intensity pattern such that, in aggregate the beams result in delivery to the patient of a 3D distribution of radiation dose that closely conforms to the shape of the target.

A wide range of strategies have been developed for planning the delivery of radiation in a way that will deliver a prescribed radiation dose to a target volume while minimizing dose to tissues outside of the target volume and in particular minimizing dose to OARs. Radiation treatment planning software that implements various ones of these strategies is commercially available. Given a radiation treatment plan it is possible to perform simulations which predict the distribution of radiation in the body of a patient when the radiation treatment plan is executed. Such simulations may be performed to validate a radiation treatment plan before irradiating a patient according to the plan.

Radiation for radiation treatment is commonly provided by a radiation treatment system such as a linear accelerator. Linear accelerators generate x-rays by bombarding a target with high energy electrons. Earlier radiation treatment systems used radioactive isotopes such as cobalt 60 as radiation sources. Modern radiation treatment systems include various interlocks and safety systems that are designed to avoid unintentionally exposing patients to radiation.

There are numerous variables that can affect whether executing a treatment plan will actually deliver the intended radiation dose to a patient. These include mechanical variables which affect accuracy of collimating or positioning the radiation beam, radiation-related variables such as the intensity profile of the beam produced by a linear accelerator or other radiation delivery system, and patient-related variables, such as the patient shape and position relative to the beam. Delivery of the treatment with acceptable spatial and dosimetric accuracy requires all these variables to be within tolerance.

The state of the art includes:
CA3014225
EP3653262
U.S. Pat. No. 8,735,828
U.S. Pat. No. 9,604,077
WO2015/118533
WO2021/007552
Lynch N, Monajemi T, Robar J L. Characterization of novel 3D printed plastic scintillation dosimeters. Biomed Phys Eng Express. 2020; 6 (5): 055014. doi: 10.1088/2057-1976/aba880
Rooney M K, Rosenberg D M, Braunstein S, et al. Three-dimensional printing in radiation oncology: A systematic review of the literature. J Appl Clin Med Phys. 2020; 21 (8): 15-26. doi: 10.1002/acm2.12907
Khosravani M R, Reinicke T. 3D-printed sensors: Current progress and future challenges. Sens Actuators Phys. 2020; 305:111916. doi: 10.1016/j.sna.2020.111916
Brivio D, Naumann L, Albert S, Sajo E, Zygmanski P. 3D printing for rapid prototyping of low-Z/density ionization chamber arrays. Med Phys. Published online 2019. doi: 10.1002/mp. 13841.
Fargher S, Steer C, Thompson L. The Use of 3D Printing in the Development of Gaseous Radiation Detectors. EPJ Web Conf. 2018; 170:01016. doi: 10.1051/epjconf/201817001016.
Albert S, Brivio D, Aldelaijan S, Sajo E, Hesser J, Zygmanski P. Towards customizable thin-panel low-Z detector arrays: electrode design for increased spatial resolution ion chamber arrays. Phys Med Biol. 2020; 65 (8): 08NT02-08NT02. doi: 10.1088/1361-6560/ab8109
Berns S, Boyarintsev A, Hugon S, et al. A novel polystyrene-based scintillator production process involving additive manufacturing. J Instrum. 2020; 15 (10): P10019-P10019. doi: 10.1088/1748-0221/15/10/P10019.
Berns S, Boillat E, Boyarintsev A, et al. Additive Manufacturing of Fine-Granularity Optically-Isolated Plastic Scintillator Elements, arXiv; 2022. doi: 10.48550/arXiv.2202.10961
Mijnheer B, Beddar S, Izewska J, Reft C. In vivo dosimetry in external beam radiotherapy. Med Phys. 2013; 40 (7): 070903. doi: 10.1118/1.4811216
Olaciregui-Ruiz I, Beddar S, Greer P, et al. In vivo dosimetry in external beam photon radiotherapy: Requirements and future directions for research, development, and clinical practice. Phys Imaging Radiat Oncol. 2020; 15:108-116. doi: 10.1016/j.phro.2020.08.003.
Robertson D, Hui C, Archambault L, Mohan R, Beddar S. Optical artefact characterization and correction in volumetric scintillation dosimetry. Phys Med Biol. 2014; 59 (1): 23-42. doi: 10.1088/0031-9155/59/1/23.
Archambault L, Briere T M, Beddar S. Transient noise characterization and filtration in CCD cameras exposed to stray radiation from a medical linear accelerator. Med Phys. 2008; 35 (10): 4342-4351. doi: 10.1118/1.2975147
Jennings M W, Rutten T P, Ottaway D J. Evaluation of the signal quality of an inexpensive CMOS camera towards imaging a high-resolution plastic scintillation detector array. Radiat Meas. 2017; 104:22-31. doi: 10.1016/j.radmeas.2017.07.004.
Hsu S H, Roberson P L, Chen Y, Marsh R B, Pierce L J, Moran J M. Assessment of skin dose for breast chest wall radiotherapy as a function of bolus material. Phys Med Biol. 2008; 53 (10): 2593-2606. doi: 10.1088/0031-9155/53/10/010
Cloutier E, Archambault L, Beaulieu L. Deformable scintillation dosimeter I: challenges and implementation using computer vision techniques. Phys Med Biol. 2021; 66 (17). doi: 10.1088/1361-6560/ac1ca1
Cloutier É, Beaulieu L, Archambault L. Deformable scintillation dosimeter: II. Real-time simultaneous measurements of dose and tracking of deformation vector fields. Phys Med Biol. Published online 2021. doi: 10.1088/1361-6560/ac1ca2.
Agency IAE. Development of Procedures for in Vivo Dosimetry in Radiotherapy. International Atomic Energy Agency; 2013.
Kry S F, Alvarez P, Cygler J E, et al. AAPM TG 191: Clinical use of luminescent dosimeters: TLDs and OSLDs. Med Phys. 2020; 47 (2): e19-e51. doi: 10.1002/mp. 13839.
Barnett E, Mackenzie M, Fallone B. IMRT point dose measurements with a diamond detector. Radiol Oncol. 2005; 39.
Andreozzi J M, Zhang R, Glaser A K, Jarvis L A, Pogue B W, Gladstone D J. Camera selection for real-time in vivo radiation treatment verification systems using Cherenkov imaging. Med Phys. 2015; 42 (2): 994-1004. doi: 10.1118/1.4906249
Ashraf M R, Bruza P, Krishnaswamy V, Gladstone D J, Pogue B W. Technical Note: Time-gating to medical linear accelerator pulses: Stray radiation detector. Med Phys. 2019; 46 (2): 1044-1048. doi: 10.1002/mp.13311.
Therriault-Proulx F, Beaulieu L, Archambault L, Beddar S. On the nature of the light produced within PMMA optical light guides in scintillation fiber-optic dosimetry. Phys Med Biol. 2013; 58 (7): 2073-2084. doi: 10.1088/0031-9155/58/7/2073

The inventors have recognized a need for practical and cost effective means for monitoring the radiation dose distribution being delivered to patients in real time during radiation treatments.

### SUMMARY

This invention has several aspects. These include, without limitation:
custom fitted radiation dosimetry apparatus;
radiation dosimetry apparatus comprising non-uniform distributions of scintillator elements;
methods for making and/or calibrating dosimetry apparatus;
systems and methods for real time monitoring of radiation dose during radiation treatment; and
3D printing apparatus.

One example aspect of the invention provides radiation dosimetry apparatus that comprises a body that supports a plurality of scintillator elements. The body has a first surface formed to conform with contours of a portion of a patient's anatomy to which radiation is to be delivered according to a patient-specific radiation treatment plan. The radiation treatment plan specifies radiation dose as a function of location on the portion of the patient's anatomy. The scintillator elements are in the body and visible on a second surface of the body opposed to the first surface. The scintillator elements comprise a material that emits scintillation light in response to the radiation specified by the radiation treatment plan.

In some embodiments the radiation treatment plan includes at least one higher dose area within which the specified radiation dose has a magnitude at least equal to a first value, at least one lower dose area within which the specified radiation dose has a magnitude that is less than or equal to a second value that is smaller than the first value; and a transition region located between the higher dose area and the lower dose area within which the specified radiation dose has values between the first value and the second value. The scintillator elements may include at least some scintillator elements in the high dose region and at least some scintillator elements in the transition region.

In some embodiments:
the scintillator elements in the transition region are of a different construction from the scintillator elements in the high dose region;
the scintillator elements in the transition region are arranged in a different pattern from the scintillator elements in the high dose region;
a density of the scintillator elements in the high dose region is greater than a density of the scintillator elements in the low dose region;
a density of the scintillator elements in the transition region is greater than a density of the scintillator elements in the high dose region; and/or
the low dose region comprises a OAR region corresponding to an organ at risk (OAR) and the scintillator elements in the OAR region have higher sensitivity to radiation than the scintillator elements in the high dose region.

In some embodiments that include an OAR region:
the OAR region has a greater density of the scintillator elements than the low dose region outside of the OAR region; and/or
the scintillation light emitted by the scintillator elements in the OAR region has a spectral composition different from the spectral composition of scintillation light emitted by the scintillator elements located outside of the OAR region.

In some embodiments at least in a neighbourhood of a boundary between the high dose region and the transition region the scintillator elements are:
elongated and oriented transversely to a line extending through the transition region from the high dose region to the low dose region;
smaller than the scintillator elements in the low dose region;
made of a different material from the scintillator elements in the low dose region;
staggered radially relative to the location of the boundary.

In some embodiments the radiation dosimetry apparatus is designed for use in a system which includes a camera positioned to image the radiation dosimetry apparatus from a camera direction and a plurality of the scintillator elements are configured to direct the scintillator light from the scintillator element preferentially in the camera direction.

In some embodiments ends of the plurality of scintillator elements facing away from the first surface are shaped to preferentially direct the scintillation light in the camera direction. For example the ends of the plurality of scintillator elements are formed to provide lens shapes and/or facets configured to cause the scintillation light to be emitted preferentially in the camera direction. For example the ends of the plurality of scintillator elements have the form of prisms and axes of the prisms are inclined relative to the first surface.

In some embodiments different ones of the scintillator elements have different sensitivities to intensity of the radiation.

In some embodiments the scintillation light emitted by different ones of the scintillator elements has different spectral characteristics.

In some embodiments the scintillation light emitted by different ones of the scintillator elements has different polarization characteristics.

In some embodiments different ones of the scintillator elements have different sizes and/or volumes.

In some embodiments different ones of the scintillator elements have different shapes.

In some embodiments the body is stiff and holds the configuration of the first surface.

In some embodiments the body comprises a thermoplastic material.

In some embodiments the body comprises a material that is transparent or translucent to the scintillation light.

In some embodiments the body comprises a material that is optically clear at least at a wavelength of the scintillation light.

In some embodiments the body comprises at least one transparent window and indicia in the window that may be aligned with a fiducial marking on the patient that is visible through the window when the radiation dosimetry apparatus is engaged against the portion of the patient's anatomy.

In some embodiments the scintillator elements are exposed on a second surface.

In some embodiments sides of the scintillator elements facing the second surface are covered with a material that is transparent to the scintillation light.

In some embodiments the body includes a light reflective layer between the scintillator elements and the first surface.

In some embodiments the body includes light reflective layers between adjacent ones of the scintillator elements.

In some embodiments the light reflective layers have reflective properties that preferentially reflect the scintillation light.

In some embodiments the scintillator elements are surrounded or partially surrounded by a material that has an index of refraction significantly lower than an index of refraction of the material of the scintillator elements such that the scintillation light is guided out of the scintillator elements at least in part by total internal reflection.

In some embodiments the radiation dosimetry apparatus comprises one or more optical elements, each of the optical elements arranged to collect the scintillation light from one of the scintillator elements and to guide the scintillation light to a location within the field of view of a camera.

In some embodiments at least one of the optical elements is removably disposed in a recess or aperture formed in the body.

In some embodiments the body has a thickness in the range of 0.8 mm to 22 mm.

In some embodiments an area of the second surface of the body is in the range of 25 cm² to 900 cm²

In some embodiments the radiation dosimetry apparatus is configured to have functionality as a patient contacting part of an immobilization device.

In some embodiments the radiation dosimetry apparatus comprises one or more members configured to hold the body fixed relative to a couch of a radiation delivery system.

Another aspect of the invention provides a system for radiation treatment. The system comprises a radiation dosimetry apparatus according to any of the embodiments described herein combined with a radiation delivery system, at least one camera arranged to image the radiation dosimetry apparatus and a control apparatus connected to receive images from the at least one camera. The control apparatus is configured to: process the images to obtain measures of the scintillation light emitted by the scintillator elements of the radiation dosimetry apparatus; determine corresponding measured radiation intensities of radiation from the radiation delivery system at the scintillation elements; compare the measured radiation intensities to planned radiation intensities provided by the radiation treatment plan; and take an action if the measured radiation intensities deviate significantly from the planned radiation intensities. In some embodiments the action includes one or more of: generating a human perceptible alarm; and stopping or pausing emission of radiation by the radiation delivery system.

In some embodiments the control system is configured to locate areas of pixels in the images, each of the areas of pixels corresponding to one of the scintillator elements of the radiation dosimetry apparatus, position a window within each of the areas of pixels and processing the images to obtain measures of the scintillation light emitted by the scintillator elements comprises summing pixel values for the pixels corresponding to each of the windows. Positioning the windows may comprise aligning a center of each window with a centroid of the area of pixels with which the window is associated.

In some embodiments the control apparatus stores separate calibration information for each of the scintillator elements, the calibration information specifying a relationship between the measure of the scintillation light emitted by the corresponding scintillator element and the measured radiation intensity at the location of the corresponding scintillator element.

In some embodiments the calibration information compensates for the geometrical relationship of the camera and the radiation dosimetry apparatus.

In some embodiments the at least one camera comprises first and second cameras and the control apparatus is configured to perform stereo processing using images from the first and second cameras.

In some embodiments: the stereo processing comprises determining a pose of the radiation dosimetry apparatus relative to the first and second cameras; the control apparatus is configured to monitoring the pose to detect motion of the radiation dosimetry apparatus; and the control apparatus is configured to take an action in response to detecting motion of the radiation dosimetry apparatus.

In some embodiments, for at least some of the scintillator elements, processing the images to obtain measures of the scintillation light emitted by the scintillator elements comprises basing the measures of the scintillation light on images captured by each of the first and second cameras.

In some embodiments the control apparatus is configured to generate a record of delivered radiation dose as a function of time for each of the scintillator elements.

In some embodiments the control apparatus is configured to update the delivered dose for each of the scintillator elements at a rate that is at least as fast as the rate at which the control apparatus receives new images from one camera of the at least one camera.

In some embodiments an OAR subset of the scintillator elements are associated with an organ at risk and the control apparatus is configured to generate a warning and/or pause or stop delivery of radiation by the radiation delivery system in response to determining that a radiation dose detected using the OAR subset of the scintillator elements exceeds a threshold.

In some embodiments the radiation delivery system is operable to emit radiation pulses and operation of shutters of the one or more cameras is synchronized to the pulses of radiation emitted by the radiation delivery system.

In some embodiments the radiation delivery system includes one or more filters operative to attenuate or remove optical noise that could otherwise be detected by the camera(s). In some embodiments the one or more filters include at least one wavelength selective filter. In some embodiments the one or more filters include at least one polarization filter. In some embodiments the one or more filters are operative to attenuate or block Cherenkov radiation.

In some embodiments the radiation dosimetry apparatus configured to function as a bolus. For example, in some embodiments the body has a thickness of at least 4 mm.

Another aspect of the invention provides a method for providing a patient-specific radiation dosimetry apparatus for use while delivering radiation to a patient according to a radiation treatment plan. The method comprises: generating a computer model for the radiation dosimetry apparatus. Generating the computer model comprises: defining a shape for a first surface of a body of the radiation dosimetry apparatus to match contours of a part of the anatomy of a patient using volumetric image data for the patient and/or optical scanning data for the patient; determining a configuration of scintillator elements to be included in the radiation dosimetry apparatus based on a distribution of radiation to be delivered to the part of the anatomy of the patient according to the radiation treatment plan; and fabricating the radiation dosimetry apparatus according to the solid model.

In some embodiments fabricating the radiation dosimetry apparatus comprises 3D printing the radiation delivery apparatus.

In some embodiments the 3D printing comprises printing the body of the radiation dosimetry apparatus using a first material and 3D printing the scintillator elements within the body using a second material that is different from the first material.

In some embodiments the 3D printing is performed using a FDM or MJF printer.

In some embodiments the method comprises, during fabrication of the radiation dosimetry apparatus, measuring scintillation light output by the scintillator elements in response to delivery of radiation to the scintillator elements.

In some embodiments the method comprises, directing ultraviolet light into the scintillation elements and measuring the scintillation light output produced by the scintillator elements in response to the ultraviolet light.

In some embodiments the method comprises, emitting the ultraviolet light from an ultraviolet light source carried by a moving element of a printer being used to perform the 3D printing.

In some embodiments the ultraviolet light source and a detector operable to detect the scintillation light are both carried by a print head of the 3D printer. In some embodiments the method comprises, in response to the measured scintillation light output by one of the scintillator elements adjusting an amount of material to be 3D printed to complete the scintillator element.

In some embodiments fabricating the radiation dosimetry apparatus comprises forming one or more recesses or pockets dimensioned to receive a dosimeter in the body of the radiation treatment apparatus.

In some embodiments the method comprises, before completing the fabricating of the radiation dosimetry apparatus, performing simulations using the solid model to estimate sensitivities of the scintillator elements of the radiation dosimetry apparatus.

Another aspect of the invention provides 3D printing apparatus. The 3D printing apparatus comprises: a print head that is movable relative to a base, the print head operable to add material to a structure being formed on the base; an ultraviolet light source mounted to the print head and oriented to illuminate a portion of the structure being formed; and a light detector carried by the print head and configured to detect scintillation light emitted by a material of the structure being formed in response to exposure to the ultraviolet light, the scintillation light having a wavelength that is different from a wavelength of the ultraviolet light.

In some embodiments the ultraviolet source comprises one or more ultraviolet light emitting diodes.

Another aspect of the invention provides a method for calibrating a radiation dosimeter apparatus comprising a scintillator element that emits scintillation light in response to exposure to radiation. The method comprises: exposing the scintillator element to ultraviolet light and monitoring the scintillation light emitted by the scintillator element in response to the exposure to a plurality of intensities of the ultraviolet light; determining a first calibration function that relates an intensity of the ultraviolet light to the amount of the scintillation light emitted by the scintillator element; and generating a second calibration function which relates an intensity of ionizing radiation at the scintillator element to an amount of the scintillation light emitted by the scintillator element by adding a correction factor to the first calibration function.

Another aspect of the invention provides radiation dosimetry apparatus for monitoring delivery of radiation to a patient. The radiation dosimetry apparatus comprises: a body having a thickness of 22 mm or less, the body formed to conform to or conformable with contours of a portion of the patient's anatomy; and a plurality of scintillator elements disposed on the body, the scintillator elements operative to emit scintillation light in response to exposure to the radiation and having transverse dimensions in a plane of the body of 5 mm or less. A number of the scintillator elements per unit area varies with position on the body and an average number of the scintillator elements per unit area is greater by a factor of at least 2 for a circular region having an area that is ⅓ of an area of the second surface of the body than for a strip that follows a perimeter of the body and has a width such that an area of the strip is ½ of an area of the second surface of the body.

Another aspect of the invention provides radiation dosimetry apparatus for monitoring delivery of radiation to a patient according to a patient specific radiation treatment plan. The radiation treatment plan specifies a distribution of radiation dose to be delivered to a portion of the patient's anatomy. The radiation dosimetry apparatus comprises: a body having a thickness of 22 mm or less, the body formed to conform to or conformable with contours of the portion of the patient's anatomy and a plurality of scintillator elements disposed on the body, the scintillator elements operative to emit scintillation light in response to exposure to the radiation and having transverse dimensions in a plane of the body of 5 mm or less. A number of the scintillator elements per unit area varies with position on the body and the average number of the scintillator elements per unit area is greater by a factor of at least 2 in those parts of the body for which the radiation treatment plan specifies a radiation dose of at least 70% of a maximum radiation dose specified by the radiation treatment plan than in those parts of the body which the radiation treatment plan specifies a radiation dose that is in the range of 15% to 45% of the maximum radiation dose specified by the radiation treatment plan.

Another aspect of the invention provides apparatus having any new and inventive feature, combination of features, or sub-combination of features as described herein.

Another aspect of the invention provides methods having any new and inventive steps, acts, combination of steps and/or acts or sub-combination of steps and/or acts as described herein.

Further aspects and example embodiments are illustrated in the accompanying drawings and/or described in the following description.

It is emphasized that the invention relates to all combinations of the above features, even if these are recited in different claims.

### BRIEF DESCRIPTION OF THE DRAWINGS

The accompanying drawings illustrate non-limiting example embodiments of the invention.

FIG. 1 is a schematic perspective view of a dosimeter apparatus according to an example embodiment if the invention.

FIGS. 1A through 1E are schematic cross section views through scintillator elements of a dosimeter apparatus.

FIG. 2A is a contour chart showing an example desired radiation dose distribution for a radiation treatment plan.

FIGS. 2B to 2D are example arrangements of scintillator elements in selected portions of a dosimeter apparatus for monitoring radiation delivery in implementing the radiation treatment plan of FIG. 2A.

FIG. 2E is a plan view showing an example arrangement of scintillator elements in a dosimeter apparatus superposed on a planned radiation dose distribution.

FIGS. 3 and 3A are perspective views showing a radiation treatment systems equipped with a real time radiation monitoring system according to example embodiments.

FIG. 4 is a functional and data flow diagram for an example apparatus for monitoring radiation intensities in real time.

FIG. 5 shows an example image processing stream for removing various artefacts from raw images.

FIG. 6 is a schematic perspective view of an example calibration system.

FIG. 7 is a flow chart for an example workflow.

FIG. 8 is a partial schematic view of portions of a 3D printer equipped with a radiation source and detector for monitoring performance of scintillator elements in a dosimeter apparatus being fabricated.

FIG. 8A is a qualitative schematic example plot of intensity of scintillation light detected as a function of position of the radiation source and detector of the apparatus of FIG. 8.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

One aspect of the invention provides apparatus useful for real time radiation dosimetry. In this context, “real-time” means that dosimetry information is available during a radiation treatment and is current, for example, to within a time that is significantly less than the total planned duration of radiation delivery for the RT treatment. The dosimetry information may, for example, be current to within a few seconds (e.g. 4 seconds or less or 2 seconds or less or 1 second or less).

The apparatus includes a body that is shaped to conform closely with a part of the anatomy of a particular patient. The body carries scintillator elements that emit light in response to the presence of radiation. The body is stiff enough to hold its shape. Engagement between the body and the anatomy of the patient may cause the body (and individual ones of the scintillator elements) to be at known positions relative to the anatomy of the patient.

The intensity of the light emitted by each of the scintillator elements is a function of the intensity of radiation at the location of the scintillator element. The light outputs of the scintillator elements may be monitored in real time (e.g. by one or more cameras) to provide a real time measure of the distribution of radiation on the patient. With appropriate dosimetric calibration, the light output of the array can be related to the dose distribution received by the patient and compared to the planned dose distribution.

Results of the monitoring may be used to one or more of:
create a record of radiation delivered to the patient during the radiation treatment;
determine whether the dose of radiation being delivered to the patient is or is not in conformance with a radiation treatment plan;
serve as a double check for other dosimeters that may be deployed.
   Advantageously the arrangement and configurations of scintillator elements may be based on the radiation treatment plan as discussed herein.

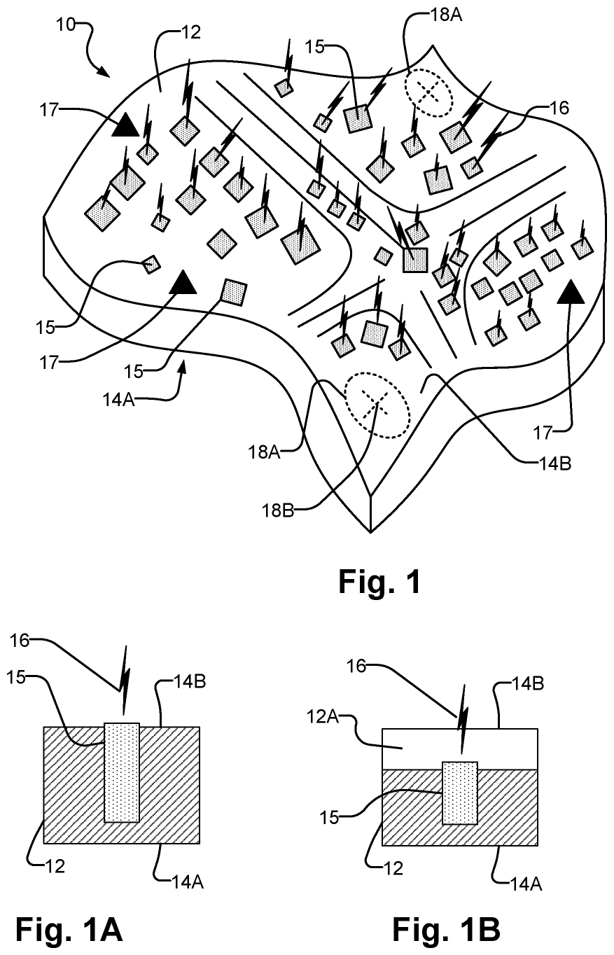

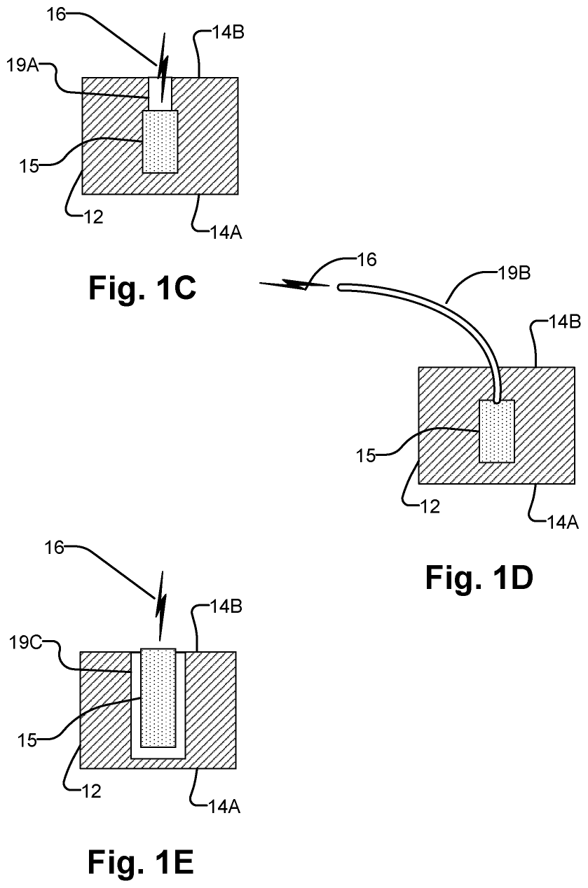

FIG. 1 shows an apparatus 10 according to an example embodiment. Apparatus 10 comprises a body 12 having a first surface 14A that is shaped to match contours of a part of a specific patient's anatomy. Body 12 may, for example, be formed from a plastic material. In some embodiments body 12 has a substantially uniform thickness.

Scintillator elements 15 are supported by and may be embedded in the material of body 12. Scintillator elements 15 emit light 16 in response to being irradiated (e.g. by high energy photons, electrons, protons or heavy ions). The light output varies with intensity of the radiation. The outer periphery of apparatus 10 may have any suitable shape including shapes that are generally rectangular, generally circular, rounded, arbitrary shapes etc.

In some embodiments body 12 is made at least partly of a material that is transparent or translucent to light 16. Making body 12 of such material tends to enhance the detection of light 16 emitted by scintillator elements 15. For example, in some embodiments body 12, or a layer of body 12 that includes a second surface 14B opposed to first surface 14A is made of a material that is optically clear at least at the wavelength of light 16 or white.

In some embodiments, body 12 is made of an optically clear material or includes optically clear windows 18A and comprises indicia 18B that may be aligned with corresponding fiducial markings (e.g. tattoo spots) that are visible on a patient. Such indicia 18B may be used to establish and/or check proper positioning of apparatus 10 on the patient for which apparatus 10 was designed.

In some embodiments apparatus 10 includes fiducial marks 17 at known locations. Fiducial marks 17 may be physical marks, e.g., printed of non-scintillating material, or scintillating material. Fiducial marks 17 may be used by image processing software to orient and scale images of apparatus 10, perform spatial transformations, and reduce intrafraction setup uncertainty. However, fiducial marks 17 are not mandatory for locating apparatus 10. Alternative approaches to determining the pose of apparatus 10 such as by optical surface imaging using structured light patterns may also be used to determine the orientation and position of apparatus 10 relative to a radiation delivery system and cameras 35 which are described elsewhere herein.

It is generally desirable to measure radiation intensity at or close to the surface of the patient's skin. If apparatus 10 is thicker than scintillator elements 15 then scintillator elements 15 may be positioned closer to first surface 14A so that the radiation that they detect more closely matches radiation present at the surface of the patient's skin.

Apparatus 10 is configured to allow light 16 to reach one or more cameras which may be located away from apparatus 10 outside of a radiation beam that is being delivered to apparatus 10. This may be achieved by one or a combination of two or more of:
positioning the scintillator elements so that the scintillator elements are exposed on a second surface 14B of apparatus 10 that is opposed to first surface 14A (see e.g. FIG. 1A);
covering scintillator elements 15 with a material 12A that is transparent to light 16 (see e.g. FIG. 1B);
including in apparatus 10 optical elements 19 (individually identified as 19A, 19B) arranged to collect light 16 from a scintillator element 15 and guide the light 16 to a location within the field of view of a camera. Optical elements 19 may, for example comprise optical fibers 19B or light guides 19A (see e.g. FIGS. 1C and 1D). Optical elements 19 may be non-removably integrated with apparatus 10 or apparatus 10 may be formed with a recess or aperture configured to receive and hold an optical element 19 in position to receive light 16 from a scintillator element and to carry the light 16 to a location at which the light 16 can be detected by a camera.

In some embodiments, apparatus 10 combines functionality of real time radiation dosimetry with some other function. For example, apparatus 10 may function also as a bolus and/or an immobilizing device or a support device.

Some RT plans call for a bolus to be placed in the radiation beam against a patient's skin. A bolus generally consists of a specified thickness of a material that is close to being tissue equivalent in interactions with radiation beams. The bolus is typically used to alter how radiation dose is distributed in the tissues of the patient. For example, a bolus may be applied to increase radiation dose delivered to the patient's skin by causing scattering of a radiation beam that is incident toward the patient. The bolus can thereby help to overcome the skin-sparing effect which occurs because radiation (especially high-energy radiation) can penetrate superficial layers of tissue while leaving relatively little radiation dose. As a result, a high energy radiation beam typically delivers a maximum dose at a distance below the skin surface. The depth at which the maximum dose occurs can be moved toward the skin surface by providing a bolus. Apparatus 10 may be made to have a thickness as required to allow apparatus act as a bolus. In some embodiments, the thickness of apparatus 10 between first surface 14A and second surface 14B is in the range of about 1 mm to a few cm.

Immobilization devices are intended to hold a part of a patient's anatomy still while radiation is being delivered to the patient. An immobilization device may comprise a sheet of radiation permeable material such as a suitable plastic that has been shaped to conform to a portion of a patient's anatomy (e.g. a face mask shaped to conform to a patient's face). The immobilization device is fixed rigidly to a couch of a radiation delivery system. Use of an immobilization device can reduce the effect of patient movements. Apparatus 10 may be configured to have functionality as a patient contacting part of an immobilization device (e.g. by including attachment points for one or more members that hold the apparatus 10 fixed relative to a couch of a radiation delivery system).

Apparatus 10 may include any desired number of scintillator elements 15 which may be arranged in any desired pattern on apparatus 10. In some embodiments scintillator elements 15 are arranged in a two-dimensional array, for example in a grid pattern. In some embodiments scintillator elements 15 are located in a pattern determined by features of a prescribed radiation dose distribution on which a RT plan is based for a specific patient.

It is not necessary that scintillator elements 15 are all the same. Scintillator elements 15 may be constructed in different ways to have different performance characteristics. For example, different scintillator elements 15 may:
have different sensitivities (i.e. the function which relates light output to radiation intensity may be different for different scintillator elements 15);
emit light having different spectral characteristics (e.g. different scintillator elements may emit light 16 of different wavelengths in response to radiation exposure);
emit light having different polarization characteristics;
have different sizes and/or volumes; and/or
have different shapes.

The distribution of scintillator elements 15 may vary across apparatus 10. For example:
scintillator elements 15 may be more densely packed in some parts of apparatus 10 and less-densely packed in other areas of apparatus 10;
the arrangements of scintillator elements 15 may be different in different areas of apparatus 10 (e.g. scintillator elements may be arranged in an array having some geometry (e.g. a square, rectangular, or triangular array) in a first part of apparatus 10 and in an array having a different geometry or in an irregular distribution in a second part of apparatus 10.

In some embodiments, one or more scintillator elements are configured to direct light 16 for optimized detection by one or more cameras 35 at known locations relative to apparatus 10. For example, scintillator elements may be configured by:
orienting the scintillator element so that the intensity of emitted light 16 at the location of a camera 35 is increased relative to other possible orientations of the scintillator element; and/or
providing features of shape on the side of the scintillator element 15 that faces second surface 15 that increase the intensity of emitted light 16 at the location of the camera 35.
locating scintillator elements on apparatus 10 at locations where the scintillator elements 15 are simultaneously visible to two cameras 35.
   The features of shape may, for example, comprise lens configurations that focus or collimate light directed toward the camera 35 and/or facets arranged to reflect toward the camera 35 light 16 that would otherwise not reach the camera 35.

Depending on the desired thickness of apparatus 10, there may be opportunity to vary the placement and thickness of scintillator elements 15 in the thickness dimension of apparatus 10. While the scintillators may be fabricated at larger thicknesses and at user defined depth within apparatus 10, practical measurement considerations require consideration. Depending on the direction from a scintillator element 15 to a camera 35, if a scintillator element is too thick light emitted within some parts of the volume of the scintillator element 15 may not be able to reach the camera 35. It is best if light 16 emitted at every point within a scintillator element 15 can reach a camera 35. Therefore, while it is possible to extend the thickness of a scintillator element 15 up to the point that the scintillator element 15 extends through the entire thickness of apparatus 10, it may be better to keep the thickness of the scintillator element 15 smaller in many cases.

If, as is often the case, radiation dose at the skin surface is not a quantity of primary interest and only a visual/quantitative representation of the position of a radiation beam and its field apertures on the patient surface is required, scintillator elements 15 may be positioned anywhere in the thickness of apparatus 10 or may extend completely through the thickness of apparatus 10.

If radiation dose at the skin surface is a primary quantity of interest, scintillator elements should be located close to the skin surface (i.e. close to first surface 14A of apparatus 10). In this case, if apparatus 10 is much thicker than about 3 or 5 mm it may be necessary to make the scintillator elements 15 thinner than the thickness of apparatus 10 so that the output of light 16 from the scintillator elements 15 is representative of skin radiation dose. These scintillator elements may be left uncovered if doing so would not cause undesirable skin dose variations or may be covered with clear or translucent material otherwise. Additionally in these cases the scintillators may be made thin and oriented parallel to first surface 14A to avoid situations where the dose measured using scintillator elements 15 is inaccurate because the radiation beam is not oriented perpendicular to the skin surface (and also to first surface 14A).

The number and locations of scintillator elements 15 is entirely customizable. Layouts for scintillator elements 15 may be informed by Monte Carlo simulations and/or treatment planning system (TPS) calculation of a patient's specific treatment plan.

In some embodiments, apparatus 10 includes layers of light reflective material that are located to increase the amount of light 16 that is emitted through second surface 14B. This is illustrated, for example in FIG. 1E which shows layers 19C of reflective material. Layers 19C may be provided between adjacent scintillator elements 15 and/or between scintillator elements 15 and first surface 14A. The light reflective material is optionally selected to preferentially reflect light 16 in comparison to other wavelengths of light. In some embodiments, light 16 has a wavelength of about 425 nm.

Apparatus 10 that includes such light reflective material may, for example, be made by FDM 3D printing in which material defining a body 12 with pockets is printed first. The pockets may subsequently be lined with a layer of reflective material and filled with a plastic scintillator material.

In some embodiments scintillator elements 15 are made of a scintillator material that has a high index of refraction and are surrounded or partially surrounded by a material with a much lower index of refraction so that total internal reflection helps to guide light 16 originating within the scintillator element 15 to exit the scintillator element 15 in a direction toward a camera 35.

It is typically desirable that apparatus 10, including body 12 and scintillator elements 15 is made of low atomic number (low-Z) materials such as suitable plastics. The materials may be selected to be compatible with a chosen manufacturing process for apparatus 10. Example materials for body 12 include rigid body thermoplastic materials such as polylactic acid (PLA), acrylonitrile butadiene styrene (ABS), polycarbonate (PC) or polypropylene (PP). Less rigid materials such as thermoplastic polyurethane (TPU), polyethylene terephthalate glycol (PETG), thermoplastic polyamide (TPA) and Nylon may also be used. Body 12 may also be composed of a thermoplastic such as PLA, ABS or Nylon doped with additional materials such as carbon fiber, glass, wood or metal. One option is to include in body 12 high Z particles to artificially modify the density, effective atomic number and consequential dosimetric properties of apparatus 10. Here, high Z particles include elements with atomic number of 12 or more. Ti, Cr, V, and Fe are examples of high Z elements.

Another option is doping the material of body 12 with reflective or absorbing pigments to modify its base reflectivity, for example to improve light output of apparatus 10 and/or to optically isolate different scintillator elements 15 from one another.

Scintillator elements 15 may, for example be made from BCF-10 scintillating fiber available from Saint Gobain Crystals. BCF-10 is a polystyrene based blue emitting plastic scintillator with a peak emission wavelength of approximately 432 nm.

A large variety of other scintillator materials are commercially available and may be used for scintillator elements 15. The material used for making scintillator elements 15 may be selected to have properties (emission wavelength, quantum efficiency, decay time etc.) that are optimized for the intended application or detector configuration. Plastic scintillators materials may also be formulated by mixing light emitting fluor into a plastic base. For example, a prepared mix of plastic and scintillating fluors may be extruded into a filament having a suitable diameter for FDM printing by an available printer. Several commercial filament making systems exist for this purpose such as the Filabot EX2™.

Plastic scintillator material commonly include a polymer containing aromatic functional groups and most typically two types of dissolved fluorescent dyes. Since the concentration of the dyes is very small (a few wt %), it is reasonable to assume that if the base plastic can be 3D printed, then the scintillator can be printed.

Some common plastics that may be used for making plastic scintillators are: Polyvinyltoluene (PVT): scintillators; and Polystyrene (PS): scintillating fibers.

In some embodiments the material used for scintillator elements 15 incorporates one or more high Z elements in a desired concentration. By selectively incorporating high Z elements in scintillator elements 15 sensitivity properties of the scintillator elements may be controlled. For example, scintillator elements 15 may be made more sensitive to low energy x-rays by incorporating a suitable concentration of high Z elements.

### Apparatus Configuration Based on Treatment Plan

The freedoms to vary the constructions and arrangements of scintillator elements 15 may be exercised to provide apparatus 10 which is specifically adapted to monitor delivery of radiation according to a particular radiation treatment plan.

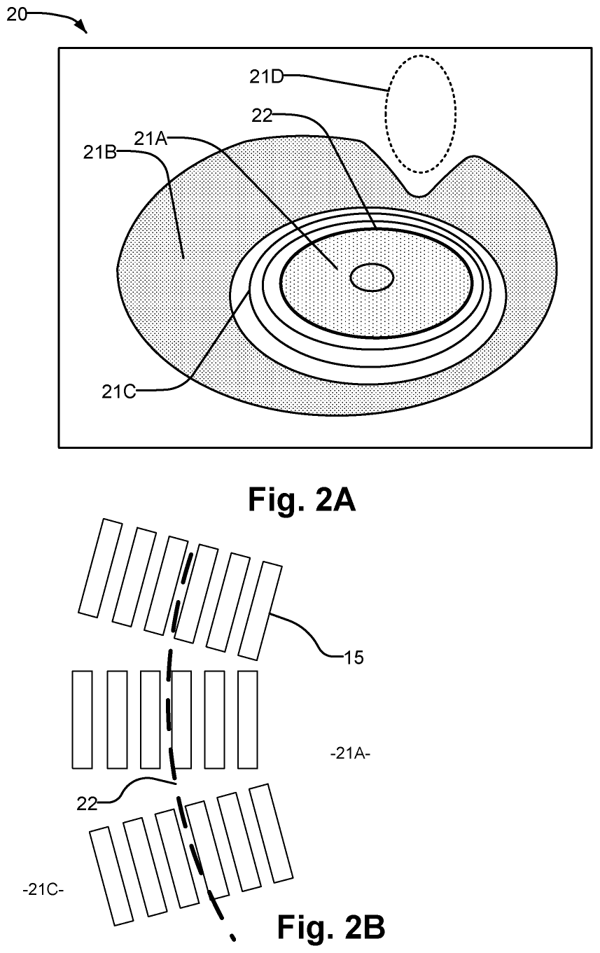

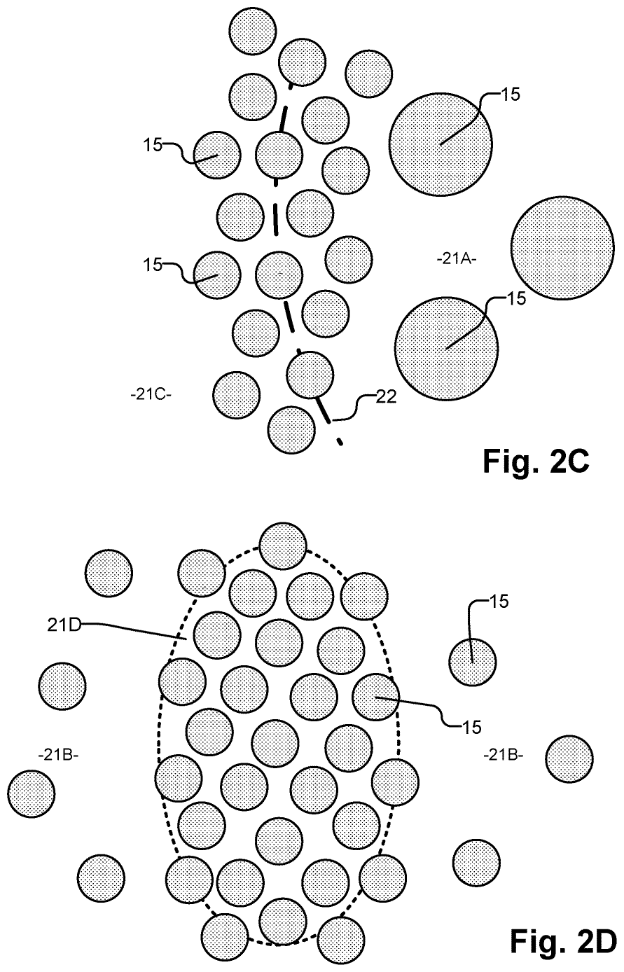

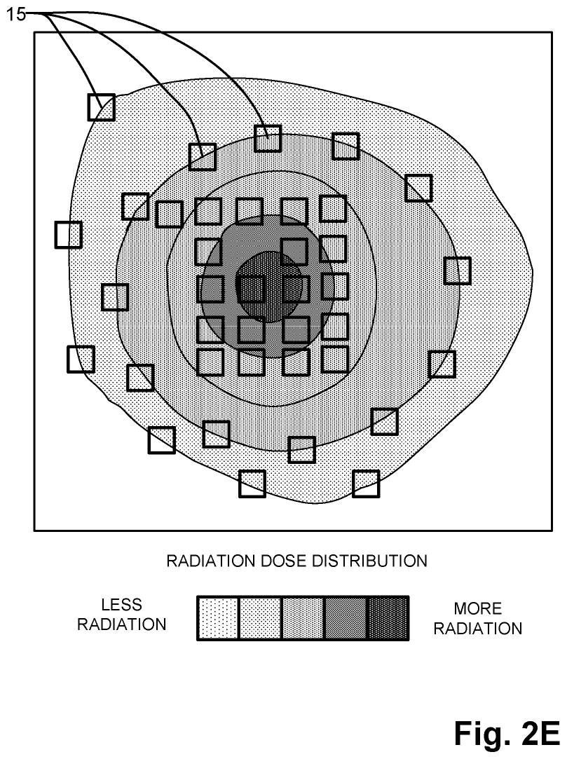

FIG. 2A depicts a contour chart 20 that shows a beam's eye view of an example planned distribution of radiation for a portion of a planned radiation treatment. Contour chart 20 includes a high intensity area 21A, a lower intensity area 21B, and a gradient or transition region 21C in which the radiation intensity should rapidly decrease from the intensity corresponding to high intensity area 21A to the lower intensity corresponding to low intensity area 21B. Also marked on contour chart 20 is an area 21D corresponding to the location of an OAR.

In delivering the radiation of contour chart 20 it is important that the boundary 22 of high intensity area 21A is in the intended location. If any part of boundary 22 is shifted outward then tissues outside of the intended location of boundary 22 will receive more radiation than desired. If any part of boundary 22 is shifted inward then tissues inside the intended location of boundary 22 will receive less radiation than desired. It is also important to minimize delivery of radiation to area 21D.

Scintillator elements 15 of an apparatus 10 may be configured to monitor the accuracy with which radiation being delivered to a patient matches contour chart 20. For example:
scintillator elements 15 in the vicinity of boundary 22 may be made narrow in a direction perpendicular to boundary 22 (FIG. 2B)—for example, scintillator elements may be elongated in a direction transverse to a gradient of the radiation dose;
scintillator elements 15 in the vicinity of boundary 22 may be made smaller than other scintillator elements 15 and/or may be made from a material for which a given volume of the material the scintillator element 15 produces more light when irradiated than the same volume of a material of which other scintillator elements 15 are made (FIG. 2C);
scintillator elements 15 in the vicinity of boundary 22 may be staggered radially relative to the location of boundary 22 (FIG. 2C).
scintillator elements 15 at different offsets relative to boundary 22 may be configured to emit light 16 of different wavelengths;
   Any of these configurations or a combination of them may provide higher positional resolution near boundary 22.

As another example:
scintillator elements 15 may be made denser (more scintillator elements per unit area) in area 21D than in other parts of low intensity area 21B (FIG. 2D);
some scintillator elements 15 located in and around area 21D may be constructed to be more sensitive (i.e. to emit more light at lower radiation intensities) than scintillator elements 15 at other locations. For example, these scintillator elements may be made from a scintillator material that emits more light 16 or more easily detectable light 16 than the scintillator material of which other ones of scintillator elements 15 are made and/or scintillator elements 15 in area 21D may be made thicker than scintillator elements 15 in other low dose areas.

As another example, regions for which a treatment plan specifies a uniform dose may have a lower density of scintillator elements 15 than other regions where the planned radiation dose is non-uniform.

As another example, regions for which a treatment plan specifies higher doses may have a higher density of scintillator elements 15 than regions for which the treatment plan specifies lower doses (FIG. 2E).

Another way to customize scintillator elements 15 is to use different materials for different scintillator elements 15 such that different ones of the scintillator elements 15 emit light 16 that is distinguishable from the light 16 emitted by other ones of the scintillator elements 15 (e.g. light 16 from some scintillator elements 15 may have different wavelengths or polarizations than light 16 from other scintillator elements 15). For example, scintillator elements that correspond to OAR may be configured to emit light 16 having a different characteristic (e.g. wavelength) than scintillator elements at other locations on apparatus 10. This may help to provide early detection of undesirably high radiation levels to OARs. In some embodiments, one (or two or more redundant) light detector(s) that are sensitive only to the wavelength of light 16 from scintillator elements associated with OARs may trigger an action if the detected light indicates that too much radiation is being delivered to the OARs.

One or more of these configurations may provide a basis for more accurate determination of the rate at which dose to an OAR corresponding to area 21D is accumulating and/or a less expensive apparatus 10 that can still reliably monitor the radiation being delivered to the patient in real time.

Apparatus 10 optionally includes one or more other types of radiation dosimeters in addition to scintillator elements 15. For example, apparatus 10 may include one or more dosimeters that can be read out after a radiation treatment is performed to measure a total radiation dose delivered: during the radiation treatment. Example types of dosimeters that may be used in this manner include thermoluminescent dosimeters (TLDs), and optically-stimulated luminescent dosimeters (OSLDs) and radiochromic film. As another example, apparatus 10 may include electronic dosimeters such as metal oxide field effect transistors (MOSFETs) or diodes which have electronic properties that change in response to radiation intensity.

In some embodiments, apparatus 10 is formed with one or more recesses or pockets each of which is shaped to received and hold a radiation dosimeter such as one of the above other types of radiation dosimeter.

### Example Real Time Radiation Monitoring System

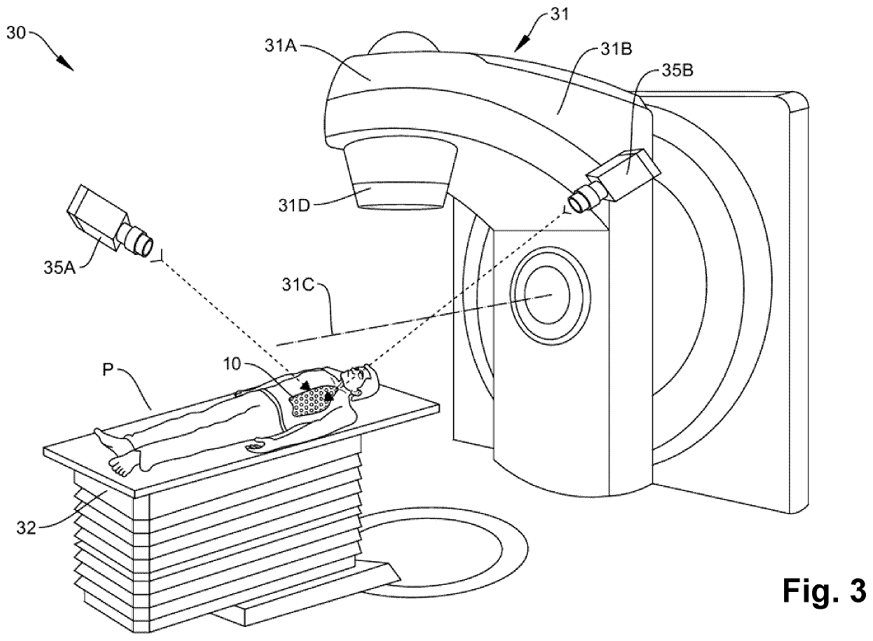

FIG. 3 illustrates apparatus 30 for delivering radiation and monitoring the delivered radiation in real time. Apparatus 30 comprises a radiation delivery system 31 such as a linear accelerator that includes a radiation source 31A that is mounted to a gantry 31B that is movable to cause radiation source 31A to rotate around an axis 31C. A collimator 31D is operable to shape a radiation beam emitted by radiation source 31A.

A patient P who is to receive RT is supported by a couch 32. In some embodiments couch 32 is movable relative to radiation delivery machine 31 in coordination with changes in the position of gantry 31B and/or changes in the configuration of collimator 31D.

An apparatus 10 is on patient P and covers a specified portion of the anatomy of patient P that will receive radiation from radiation source 31A. Apparatus 10 is formed to conform with the specified portion of the anatomy of patient P against which apparatus 10 is placed.

At least one camera 35 is positioned to view apparatus 10. In apparatus 30, first and second cameras 35A and 35B (generally or collectively cameras 35) are each positioned to obtain images of apparatus 10. Cameras 35 are each able to detect light 16 emitted by scintillator elements 15 as a result of scintillator elements 15 being exposed to radiation from radiation source 31A. Cameras 35 may have monochrome or colour light sensors. If cameras 35 are colour cameras then colour filtering may be applied to distinguish light 16 from different scintillators (that emit light 16 of different wavelengths) and/or to distinguish light 16 from background/unwanted light.

Cameras 35 may be located in any of a variety of locations from which there is clear visibility of apparatus 10 and the cameras do not interfere with delivery of RT to patient P. Cameras 35 may, for example be mounted to the ceiling or wall of a treatment room, on a stand attached to the foot of couch 32 (FIG. 3A), on a stand fixed to the floor of the treatment room or the like. Mounting cameras 35 to couch 32 is beneficial in cases where couch 32 is moved as RT is performed because cameras 35 and apparatus 10 can remain in the same relative poses as couch 32 is moved. If apparatus 10 is moved relative to cameras 35 during treatment then cameras 35 may be moved and/or image processing may be performed to track motions of scintillator elements 15 in images acquired by cameras 35.

In some embodiments, apparatus 30 includes one or more filters to reduce noise. One source of noise is Cherenkov radiation. System 30 may, for example, incorporate one or more wavelength and/or polarizing filters which remove or reduce noise (from Cherenkov radiation and/or other noise sources). Such selective filtration can mitigate the impact of extraneous light sources and improve the signal-to-noise ratio.

Filter(s) may be provided in the optical path between scintillator elements 15 of apparatus 10 and detectors of camera(s) 35. In some embodiments the filters are integrated with cameras 35 and/or lenses of cameras 35. Filters 36 are shown schematically in FIG. 3A.

The filters may, for example, comprise one or more polarizing filters and/or one or more wavelength selective filters. For example, wavelength selective filters may be configured to transmit to detectors of camera(s) 35 wavelengths of light that are emitted by scintillator elements 15 of apparatus 10 in response to radiation from radiation source 31A and are desired for monitoring radiation dose to patient P. The filters may block and/or attenuate light of other wavelengths. For example, the wavelength selective filters may be operative to block or attenuate Cherenkov radiation. The wavelength selective filters may, for example, comprise band-pass filters which pass wavelengths of light that are used to measure radiation dose at scintillator elements 15 and/or band-stop filters which block wavelengths corresponding to Cherenkov radiation or other noise.

As another example, polarizing filters selectively pass light with a specific polarization orientation and attenuate or block light having other polarizations. The filters may include one or more polarizing filters oriented to attenuate or block light having polarizations corresponding to Cherenkov radiation and/or other noise sources.

During delivery of RT, camera(s) 35 obtain images of apparatus 10 and detect light 16 originating from scintillator elements 15. The amount of light 16 detected by a camera 35 from each scintillator element 15 is related to the intensity of radiation incident on the scintillator element 15 by a function that is typically different for different ones of scintillator elements 15 and also for different cameras 35. The functional relationship between radiation intensity and detected light output for each scintillator element 15 may be determined by calibrating apparatus 10 as described below. This functional relationship typically depends on all of the material and construction of individual scintillator elements 15, the relative positions and orientations of camera(s) 35 and apparatus 10 and properties of cameras 35.

In some embodiments, the possibility that ambient light could interfere with detection of light 16 is reduced by dimming ambient lighting, delivering ambient lighting using light sources that do not emit significant amounts of light in a band of wavelengths that includes the wavelength(s) of light and/or applying a filter which passes light 16 to cameras 35 but blocks light of other wavelengths.

Cameras 35 may be rigidly fixed such that their fields of view remain fixed relative to radiation delivery system 31. Where system 30 includes two spaced apart cameras 35, alignment of the cameras may be checked to ensure the cameras 35 are in proper alignment and their depth measurements are accurate, for example by imaging a calibration check board that is placed at a known location and orientation relative to radiation delivery system 31. The check may involve determining the locations of calibration points on the calibration check board by stereo processing and comparing the determined locations to known (e.g. previously determined) locations of the calibration points. A match indicates that cameras 35 remain properly aligned.

When the relationship between detected light 16 and radiation intensity is known for a particular scintillator element 15 the radiation intensity at the location of the scintillator element may be determined in real time based on the known relationship. The radiation dose at the location of the scintillator element 15 may be determined by integrating the measured radiation intensity over time.

Scintillator elements 15 at locations where radiation intensity is low emit light 16 that is less intense than light 16 emitted by scintillator elements 15 located where the radiation field is more intense. At sufficiently low radiation intensities it can become difficult to distinguish light 16 from ambient light. Some radiation delivery systems 31 emit radiation in the form of a rapid sequence of pulses. In some embodiments, detection of light 16, particularly for low radiation intensity levels is optimized by synchronizing operation of the shutters (typically electronic shutters) of cameras 35 to the pulses of radiation emitted by radiation delivery system 31 so that cameras 35 capture light 16 during a period which includes a maximum number of the pulses for the frame rate of cameras 35.

Apparatus 10 optionally includes one or more optical fibers that are each arranged to carry light 16 from one scintillator element 15 to an optical detector (which may be located outside of a radiation field). Light 16 from a scintillator element 15 that is detected by the optical detector may be compared to light from the scintillator element 15 detected by a camera 35 to check for proper operation of system 30. A few such optical fibers/detectors may be sufficient to detect problems such as a change in position of a camera 35 or apparatus 10.

### Example Processing Apparatus and Methods

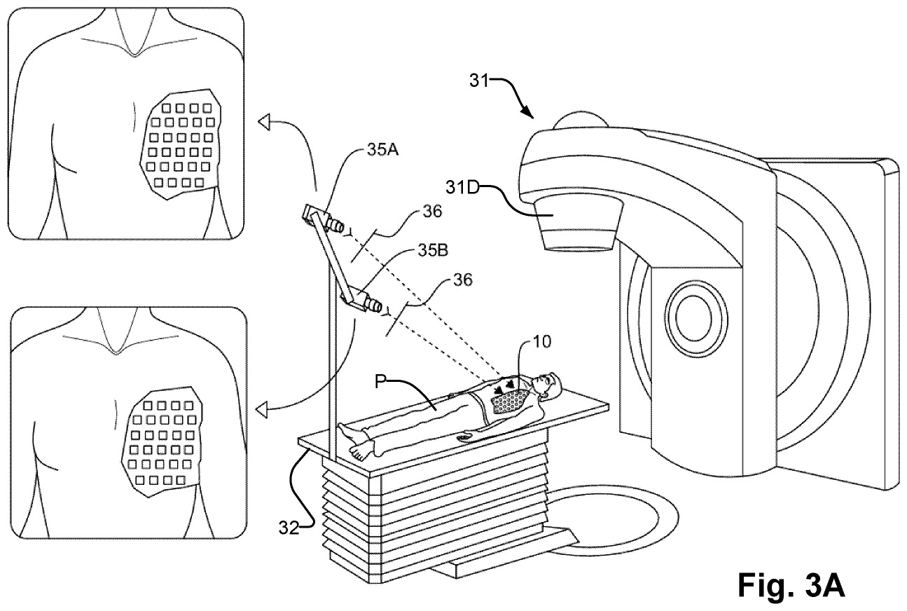

FIG. 4 is a functional block diagram for an example apparatus 40 which is operable to process images from camera(s) 35 and to take one or more of various actions depending on results of the processing. Apparatus 40 and variations of apparatus 40 may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in apparatus 40 may implement methods as described herein by executing software instructions in a program memory accessible to the processors.

The invention may also be provided in the form of a program product. The program product may comprise any non-transitory medium which carries a set of computer-readable instructions which, when executed by a data processor, cause the data processor to execute a method performed by apparatus 40. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMS, hardwired or preprogrammed chips (e.g., EEPROM semiconductor chips), nanotechnology memory, or the like. The computer-readable signals on the program product may optionally be compressed or encrypted.

Signals encoding images 42A and 42B, respectively from cameras 35A and 35B, are received at interface 41. Depending upon the spatial resolution and frame rate of cameras 35 the rate at which image data is received at interface 41 may be very high. In some embodiments, interface 41 comprises a high speed data interface such as a dedicated PCIe interface. In some embodiments, cameras 35 are configured to not transmit some non-essential data to interface 41. For example, instead of sending pixel values for every pixel of an image, camera(s) 35 may be configured to not transmit some or all pixel values that correspond to pixels that do not correspond to any of scintillator elements 15. Cameras 35 may, for example comprise integrated systems (e.g. configured field programmable gate arrays) configured to select and transmit pixel values for pixels that correspond to scintillator elements 15.

Images 42A and 42B are processed at block 44 to measure an amount of light 16 represented in the image 42A or 42B for each scintillator element 15 of apparatus 10. The processing of block 44 may, for example use data 43A and 43B that respectively record which pixels of each image 42A or 42B are associated with each scintillator element 15. It is generally considered beneficial that at least 100 pixels of each image 42 (e.g. a 10×10 block of pixels) corresponds to each of the scintillator elements 15 that are visible in the image 42. In block 44, pixel values for the pixels associated with each scintillator element 15 in each of images 42 are combined (e.g. by summing) to yield a light intensity value 45 representing the amount of light 16 detected from the corresponding scintillator element 16.

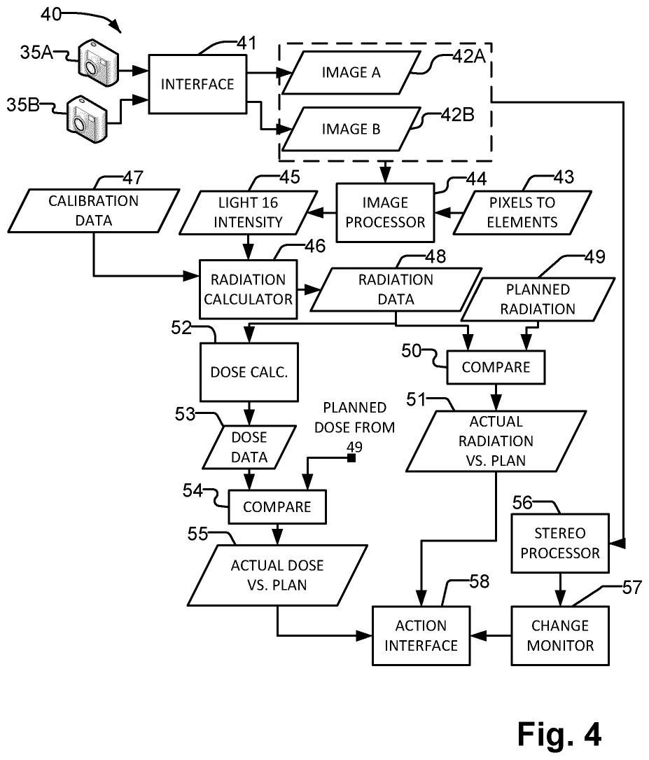

Block 44 may include performing camera-related image corrections such as vignetting, and compensating for distortion. FIG. 5 shows an example image processing stream for removing various artefacts from raw images received from camera(s) 35.

In some embodiments, block 44 includes processing to remove imaging artefacts. Examples of imaging artefacts that block 44 may correct for and examples of approaches for correcting for such imaging artefacts are provided elsewhere herein.

Where calibration of apparatus 10 does not already take into account the distances from scintillator elements 15 to a camera 35 block 44 may perform a source-to-camera distance correction. The source-to-camera distance correction may for example include multiplying the amount of detected light 16 at each pixel of camera 35 that corresponding to a scintillator element 15 by a factor that includes r² where ris the distance from the scintillator element 15 to the camera 35 (inverse square correction). If the number of pixels that are used to measure the light output associated with each scintillator element 15 is distance dependent then additional or different corrections may be required to adjust for the different number of pixels that may be used to determine the output of light 16 for different scintillator elements 15.

In some embodiments, two cameras 35 that have a known spatial relationship to apparatus 10 are used to image apparatus 10. Images from the cameras may be processed using stereoscopic imaging algorithms to determine distances of each scintillator element to one or both of the cameras 35. These distances may be used as a basis for source-to-camera distance correction.

The distances from scintillator elements to camera(s) 35 may be accounted for in calibration information. For example, calibration of apparatus 10 may be performed using a calibration setup that has the same geometry as the setup used while monitoring RT of a patient P.

Another option is to use an optical or other 3D scanning technology to measure the orientation and position of apparatus 10 on a patient P when apparatus 10 and patient P are positioned in the same way as for receiving a planned RT treatment. Results of the scanning may be used (optionally in combination with a 3D model of apparatus 10) to determine the distance from each scintillator element 15 to one or more cameras 35. Inverse square corrections may then be applied based on these distances.

In some embodiments, block 44 takes into account differences in the light output of scintillator elements 15 with direction. For example, in at least some cases Cerenkov light can form a significant component of light 16 and for at least some radiation that may be used in RT, the intensity of Cerenkov light may have a non-negligible dependence on direction and beam energy. Block 44 may correct for such dependence by applying a correction based on a difference between the direction of a radiation beam directed onto apparatus 10 and the direction between a specific scintillator element 15 and a camera 35. The correction may be based, for example, on empirical measurements and/or theoretical calculations of the direction dependence of Cerenkov light for the particular radiation to be used to deliver RT to a patient P.

Block 46 performs calculations which take light intensity values 45 as inputs and produce as outputs the radiation intensity at each scintillator element 15. The calculations of block 46 may, for example process a measured intensity of light 16 detected at a camera 35 from a scintillator element 15 (an intensity value 45) and use calibration information to determine the corresponding radiation intensity at the scintillator element 15. Calibration information 47 may, for example, be stored in one or more lookup table (LUT), table, function, parameters of a parameterized function or the like. In some embodiments, block 46 comprises inputting light intensity values 45 into a parameterized function which outputs radiation intensity data 48. In such embodiments calibration information 47 may comprise a calibration table that includes a set of parameters for each camera for each scintillator element. The parameters of the calibration table may be determined in a calibration routine as described below. Block 46 may save radiation intensity data 48 in a data store for future reference.

The radiation intensity for any scintillator element 15 may be determined using an image 42 from one camera 35, as described above. If apparatus 30 includes two or more cameras 35 that each obtain images of a particular scintillator element 15 of apparatus 10 then block 46 may be configured to compute the radiation intensity at the particular scintillator element 15 based on radiation intensities determined by processing images 42 from two or more such cameras. Since the field of view of each of the cameras and the distances of individual scintillator elements 15 to different ones of the cameras 35 will in general be different separate calibration data is required for each camera 35. Radiation intensities of the same scintillator element 15 determined by two or more cameras may be combined, for example by averaging or weighted averaging to obtain a potentially more accurate radiation intensity.

Block 50 compares the radiation intensities 48 determined by block 46 to corresponding values in a table 49. The comparisons (comparison data 51) indicate whether any of the detected radiation intensities deviate significantly from expected values for the radiation intensities. Table 49 may, for example comprise high and low thresholds for the radiation intensity at each scintillator element. Different thresholds may be provided for different sections of a planned RT treatment.

Block 52 maintains a running set of estimates of radiation dose (dose data 53) that has been delivered so far to patient P during a planned RT treatment at locations of scintillator elements 15. Block 54 compares dose data 53 to planned dose data from 49 to yield data 55 indicating actual vs planned doses.

Block 56 is a stereo processing block that processes images 42A and 42B to determine locations or poses (locations and orientations in 3D space) of scintillator elements 15 and apparatus 10 relative to cameras 35A and 35B.

Block 57 checks for changes in the locations or poses determined by block 52.

Block 58 takes actions based on results provided by blocks 50 and/or 54 and/or 57. Examples of possible actions include:
1. pausing or performing an emergency stop of the RT;
2. operating a warning device (light, indicator, sound, tactile warning);
3. slowing delivery of radiation.
   One or more of these actions may be taken at block 58 in response to one or more of: block 50 having detected radiation intensities for one or more scintillator elements that are too high or too low; block 54 having determined a cumulative radiation dose that is too high or too low; a pattern of the radiation intensities determined by block 52 does not sufficiently match a planned pattern of radiation intensities; and block 57 detects changes in pose or position of apparatus 10 of more than a threshold amount.

In some embodiments apparatus 40 is configured to compute a dose map of radiation delivered to patient P in the course of a RT treatment. This may be done, for example, by time integrating radiation intensities detected at scintillator elements 15. FIG. 4 includes block 52 which computes a delivered dose. Block 52 may update a delivered dose by scintillator element each time a new image 42 is received. Block 52 may output dose as a function of location (scintillator element 15) and time.

In some embodiments apparatus 40 is configured to monitor accumulated dose and to take action if the accumulated dose falls outside of an acceptable range. For example, action may be taken if a measured accumulated dose for a scintillator element in a high dose region deviates from a corresponding expected dose by more than a threshold amount. The threshold may be adjustable depending on the treatment type or application. If the measured dose exceeds the expected dose by more than the threshold (e.g. by more than 5%) apparatus 40 may generate a warning signal to a responsible radiotherapist administering the treatment, or control radiation delivery system 30 to pause beam delivery.

In some embodiments, block 52 operates by summing pixel values for processed images from block 44. An entire radiation treatment typically produces on the order of about 100 to about 600 images where the images are obtained at 5 frames per second. The pixel values of a series of images 42 may be summed to produce a final image representing the entire dose delivery. Pixel values in the final image are indicative of total dose received at the corresponding scintillator element 15.

Because the signal (light 16) produced by scintillator elements 15 is substantially higher than the remaining background following processing, a binary image mask can be produced using a grayscale level (GSL) threshold. The binary image mask may be processed to identify a group of pixels that represent light 16 received from each scintillator element 15.

Images of different scintillator elements acquired by cameras 35 can have different areas (different numbers of pixels) because the scintillator elements may have different sizes, different scintillator elements may be at different distances from the camera 35 and different scintillator elements may have different orientations relative to the camera 35. In some embodiments, all pixels in an image that correspond to a scintillator element 15 are used to measure the light 16 output by the scintillator element 15. In other embodiments only some of the pixels in the image that correspond to the scintillator element are used to measure the output of light 16 by the scintillator element. For example, in some embodiments, a window of a standard size and shape is used to identify a group of pixels corresponding to each scintillator element 15. The pixels within the window are used to measure the output of light 16 from the scintillator element. The size and shape of the window can be selected so that the window is small enough to fit within the area of the image corresponding to any depicted scintillator element 15. For example, in some embodiments the window may be chosen to have a square or rectangular configuration. Example window sizes are 10 pixels by 10 pixels or 25 pixels by 25 pixels. The window may be placed consistently relative to the portion of the image corresponding to each scintillator element 15. For example, the center of the window may be aligned with the center or centroid of the portion of the image corresponding to each scintillator element 15.

For example the image mask may be processed to locate the center of the image of each scintillator element 15 in the final image. These center locations may be used to select pixels that represent the light 16 output by each scintillator element 15. For example, MATLAB Blob Analysis may be used to locate the center of the group of pixels corresponding to each scintillator element 15 in the mask. In an example embodiment a window of a selected size (e.g. a 25×25-pixel window) in the final image is selected for each scintillator element. The light output for each scintillator element 15 is then determined by calculating the total light output in GSL of the pixels that lie within the corresponding window.

### Calibration

Apparatus 10 may be calibrated by delivering known amounts of radiation to scintillator elements 15 and measuring the resulting output of light 16 from each scintillator element 15 that is detected by a camera 35. The radiation used for calibration may have the same energy spectrum as the radiation to be used for the planned RT treatment. If the radiation used for calibration has a different energy spectrum from the radiation to be used for the planned RT treatment then known response relationships may be used to compensate for the differences in the energy spectra of the radiation used for calibration and treatment.

Since the amount of light 16 detected by a camera 35 depends in part on the relative positions and poses of camera 35 and apparatus 10 it is preferable to perform calibration with cameras 35 and apparatus 10 positioned in the same relative positions that they will be in during delivery of RT. It is also possible to perform calibration of apparatus 10 by measuring the amount of light 16 emitted by each scintillator element 15 when exposed to radiation of known intensity and then adjusting the calibration to account for the geometry of camera(s) 35 that will be used while monitoring delivery of radiation to a patient (geometric corrections may be applied to account for the distances of camera(s) 35 from individual scintillator elements 15, as well as orientation of each scintillator element 15 to the direction from which radiation is incident on the scintillator element and the direction from the scintillator element 15 to a camera 35).

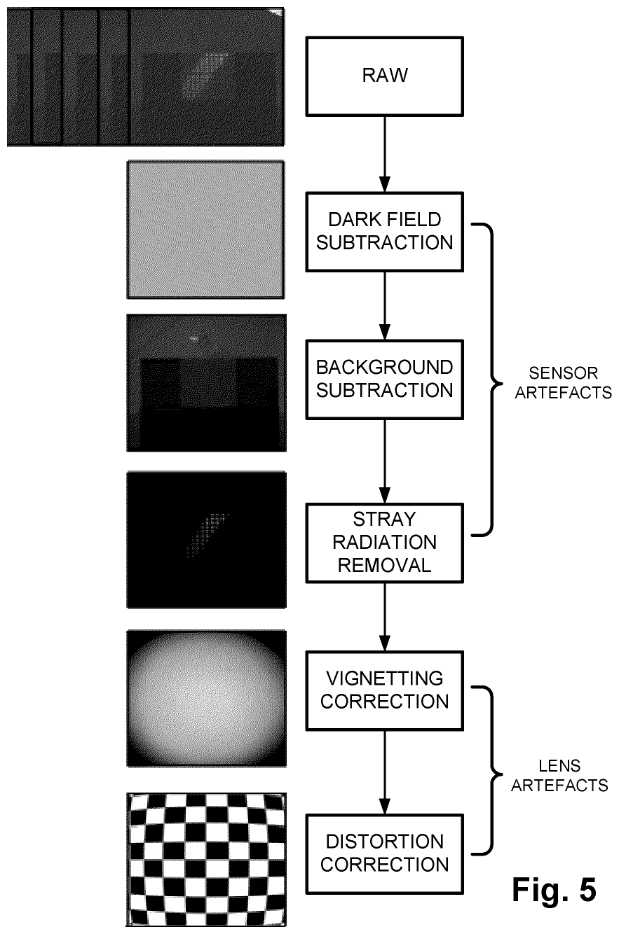

In some embodiments, calibration is performed on a calibration fixture. An example calibration fixture 60 is shown schematically in FIG. 6. Calibration fixture 60 includes support(s) s 61 for camera(s) 35, a support 62 for apparatus 10 and a radiation source 63. In some embodiments, apparatus 10 is supported for calibration adjacent to a volume 64 of tissue equivalent material such as Solid Water™.

Calibration may be performed using calibration fixture 60 by: supporting apparatus 10 on support 62; and delivering radiation to apparatus 10 at a range of intensities. For each of the radiation intensities the calibration includes: monitoring the amount of light 16 detected by one or more camera(s) 35 supported on support(s) 61 for each scintillator element 15; and determining the radiation intensity at each of the scintillator elements 15 of apparatus 10. The radiation intensity at each of scintillator elements 15 may be determined by simulation, for example Monte Carlo simulation or calculations made using a radiation treatment planning system (TPS), based on the known intensity of radiation incident on apparatus 10 and the known properties of the materials of apparatus 10 and the tissue equivalent material of volume 64.

A relationship between detected light 16 and radiation intensity may be determined one or more camera(s) 35 for each scintillator element 15. This relationship may, for example, be embodied in calibration information 47 which may, for example, take the form of one or more lookup tables (LUTs), tables, functions, parameter(s) for a parameterized function or the like.

In an example embodiment, each scintillator element 15 of apparatus 10 is calibrated to dose for future measurements using a unique calibration coefficient Ccal determined from the following relationship:

$${S}_{M} \times {C}_{c a l} = {S}_{M} \times ( \frac{{D}_{C a l}}{{S}_{C a l}} ) = {D}_{M}$$

Where S_{M} is the corrected total light output of a given scintillator element 15 in GSL, S_{Cal} is the corrected total light output of the same scintillator element 15 in the calibration geometry in GSL, D_{Cal} is the corresponding dose in cGy determined by Monte Carlo simulation in the calibration geometry and D_{M} is the final dose measurement in cGy.

In some embodiments, apparatus 10 is calibrated using a different type of radiation than will be used in RT for a patient P. For example, in some embodiments radiation source 63 is a source of ultraviolet light such as an ultraviolet lamp, one or more ultraviolet-emitting LEDs or ultraviolet-emitting laser diodes or the like. In such embodiments the calibration may include a known function that specifies the relationship between the output of light 16 for a scintillator element 15 when illuminated by the ultraviolet light and the output of light 16 for the same scintillator element 15 when the same scintillator element 15 is exposed to a particular intensity of the radiation that will be used to treat patient P. This function may be determined experimentally.

The ultraviolet light may, for example have a wavelength in the range of about 265 nm to about 310 nm. In some embodiments the ultraviolet light source has a peak irradiance of at least 50-90 mW/cm² at 2 mm. In some embodiments the ultraviolet light is collimated to have a shape such as a narrow cone to minimize its width at the measurement location to the diameter of a single scintillator element.

In some embodiments an ultraviolet light source is positioned to illuminate scintillator elements 15 one at a time. The ultraviolet light source may, for example, be positioned using a positioner such as a robot arm. The known geometry of apparatus 10 may be used to determine positions and orientations of the ultraviolet light source such that the ultraviolet light source is held to illuminate each scintillator element 15 from a known distance and with a known alignment relative to the scintillator element 15. The ultraviolet light source may be held such that the distances and/or alignment of the ultraviolet light source is the same for each of scintillator elements 15. In some embodiments a detector operable to detect light 16 is positioned by the same positioner that carries the ultraviolet light source.

As UV radiation does not possess the same penetrating power as X-rays the UV light should be incident on the visible exterior surface of a given scintillator element 15. Ideally, the direction of incidence is aligned with an axis of the scintillator element 15 (eg. from directly above the scintillator element 15). The orientation of the light source should remain consistent such that emitted ultraviolet light is incident on the scintillator element 15 at an angle which results in excitation of substantially the full volume of the scintillator element 15. In some embodiments ultraviolet light is directed at the scintillator element 15 from one angle and resulting light 16 may be detected at a different angle chosen so that ultraviolet excitation light does not reach the light detector used to detect the scintillation light 16. It is also possible to provide a filter to reject the ultraviolet excitation light from the detector.

Using ultraviolet light to calibrate apparatus 10 advantageously does not require access to a linear accelerator or other source of the same radiation that will be delivered for RT of patient P and may also be safer for personnel performing the calibration.

In some embodiments a series of calibration images of apparatus 10 are taken while apparatus 10 is in position for delivering RT to a patient. The calibration images are taken while apparatus 10 is illuminated with UV light. Since the UV light causes scintillator elements 15 to emit light 16, such images can be processed to generate an image mask that indicates positions of each of scintillator elements 15 in the field of view of a camera 35. Stereo image processing may be applied to two such masks obtained by two cameras 35 to determine the locations of each scintillator element 15 relative to the cameras 35. By using UV light to cause scintillator elements 15 to emit light 16, positions of scintillator elements 15 relative to cameras 35 may be determined without ionizing radiation. If stereoscopic images are acquired with the UV-illumination technique, the position of the printed array relative to the isocenter of the linear accelerator can be calculated and evaluated for correctness (based on the planned position of patient P and apparatus 10 for a planned RT treatment).

### Methods for Making Apparatus Comprising Scintillator Arrays

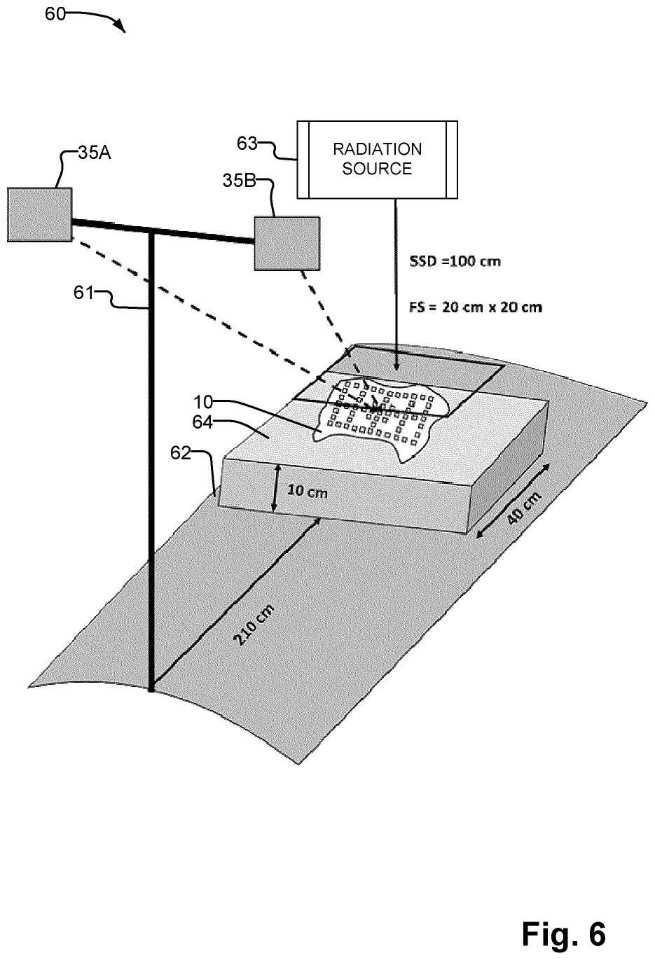

An example workflow 70 within which making of apparatus 10 may be performed is shown in FIG. 7.

The contours of first surface 14A may be based on contour information 71 which may, for example, be determined from volumetric image data for of patient P (e.g. computed tomography (CT) or magnetic resonance imaging (MRI) images from which a surface profile for a portion of the anatomy of patient P against which apparatus 10 will be placed in use can be determined) and/or by scanning the appropriate part of the patient's anatomy with an optical or other scanner that outputs surface contours.

A 3D computer model of apparatus 10 may be designed (e.g. at S72) in which the contours of first surface 14 are defined by the outer contours of a selected part of the anatomy of patient P against which apparatus 10 will be placed while in use. The model may also include scintillator elements 15. The sizes, shapes etc. of scintillator elements 15 may be designed with reference to a radiation treatment plan 73 for patient P as described elsewhere herein. Different scintillator elements may be designed to have different responses to radiation based on a patient-specific geometry/radiotherapy plan. For example, scintillator elements 15 at locations where the radiotherapy plan calls for lower radiation intensities may be designed to be more sensitive (higher output of light 16 for the same radiation intensity) than scintillator elements at locations where the radiation treatment plan calls for greater intensity of radiation.

If apparatus 10 is intended to provide functionality in addition to serving as a dosimeter (e.g. functionality as a bolus and/or as an immobilization device) then the 3D model may incorporate features to support the additional functionality (e.g. a desired thickness for a bolus and/or suitable attachment points for an immobilization device).

The 3D model may be designed using computer aided design (CAD) software. In some embodiments the scintillator elements 15 and the body of apparatus 10 are initially modeled as two separate components. The first component defines the body composed of a suitable material such as PLA configured with voids to receive scintillator elements 15. The second component defines a corresponding set of scintillator elements 15. These components may then be imported into and combined in suitable software. The 3D model may include removable support structures that support apparatus 10 during 3D printing.

Apparatus 10 may, for example, be made using an additive manufacturing process such as 3D printing (e.g. at S73). Advantageously additive manufacturing may be used to fabricate both scintillator elements 15 and the remainder of the body 12 of apparatus 10. This may be achieved, for example, by using a 3D printer designed for 3D printing structures that include plural different materials. For example, some 3D printers include plural nozzles that may each be applied for adding a different material to a 3D printed structure. Multi Jet Fusion (MJF) is another 3D printing process that may be used to fabricate apparatus 10.

To prepare for 3D printing the 3D model may be “sliced” according to print parameters to yield a set of 3D printer instructions (e.g. g-code) which may be executed by a 3D printer to print apparatus 10. An example of slicer software is Cura™ software available from Ultimaker.

In some embodiments, a-priori knowledge of the design of apparatus 10 (e.g. a 3D model of apparatus 10) is used to simulate operation of apparatus 10 (S74). The results of the simulation may be applied (e.g. at S75) to provide an initial calibration of apparatus 10. For example: where scintillator elements are of different shapes and/or sizes, computer modelling may be performed to estimate the output of light 16 for each scintillator element 15 as a function of radiation intensity. Such an initial calibration may be used, for example, to assess whether or not the planned radiation intensities are likely to be within the detection ranges of the scintillator elements of the proposed arrangement of scintillator elements. If not, adjustments may be made to the 3D model (e.g. by increasing volumes or forms of scintillator elements for which the initial calibration indicates that the output of light 16 is likely to be too small to detect with a desired accuracy).

By leveraging existing a priori information contained in the CAD model, simulation(s) to evaluate the expected performance of apparatus 10 (e.g. Monte Carlo simulations) can be performed in advance of and/or in tandem with fabrication of apparatus 10. The results of the Monte Carlo simulation may be used for initial array sensitivity correction and dose calibration.

After apparatus 10 has been fabricated apparatus 10 may be calibrated (e.g. at S76A, S76B and S76C). Subsequently, apparatus 10 may be applied for real time dosimetry (e.g. at S77).

In a clinical setting, a workflow like workflow 70 can drastically reduce the overall preparation time required prior to performing patient-specific in-vivo dosimetry with 3D printed scintillator arrays, as calibration of the array may be commenced even before fabrication of apparatus 10 has been completed. Furthermore, this workflow can efficiently accommodate off-site manufacturing of apparatus 10 since preparations for calibrating and using apparatus 10 may be performed in parallel with the manufacturing of apparatus 10.

Estimates of light output based on the designed shapes and sizes of scintillator elements may not be as accurate as desired for some applications due to process variations. The inventors have found that there can be significant variations in the output of light 16 among different scintillator elements printed by the same printer using the same materials and same instructions.

Process variations in the production of apparatus 10 may be at least partially compensated for by performing in-process calibration during fabrication of apparatus 10. The in-process calibration may, for example, comprise illuminating completed or partly-completed scintillator elements 15 with a set intensity of radiation which causes emission of light 16 from the scintillator elements and monitoring the intensity of the emitted light 16. In some embodiments the radiation used to stimulate emission of light 16 from scintillator elements during fabrication of apparatus 10 is ultraviolet light. In some embodiments an ultraviolet light source and a camera or other light detector operable to detect emission of light 16 from scintillator elements are integrated with a 3D printer so that performance of scintillator elements of an apparatus 10 being fabricated may be assessed without removing the partially completed apparatus 10 from the printer.

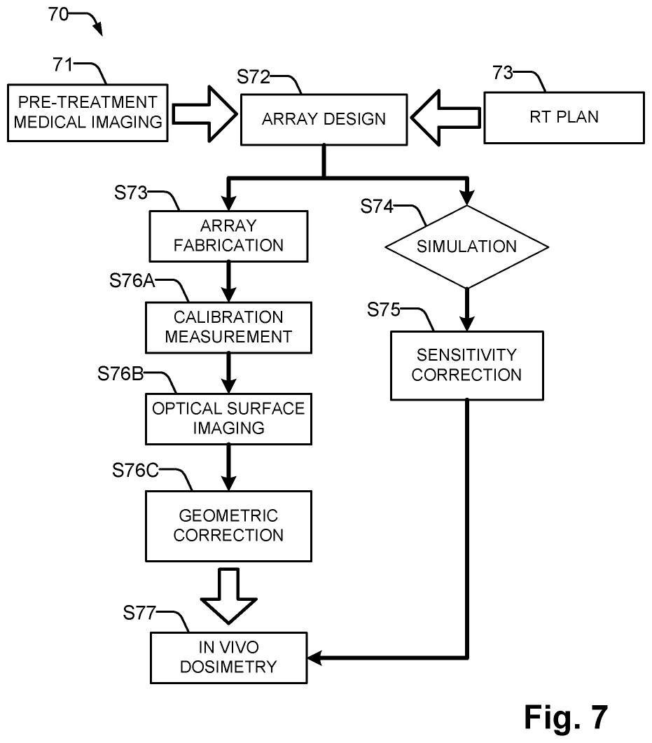

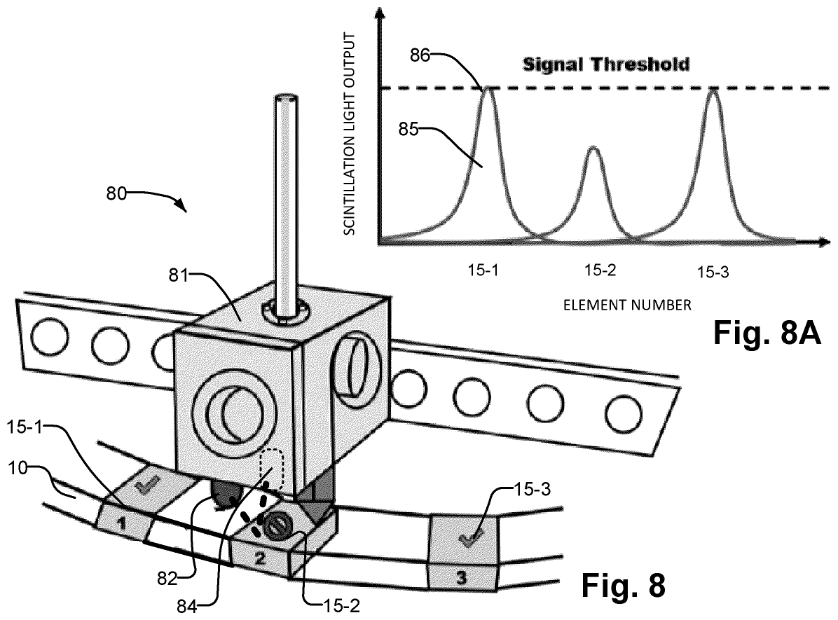

FIG. 8 is a schematic illustration showing portions of an example 3D printer 80 having a print head 81 that is operable to print an apparatus 10. Printer 80 includes a UV light source 82 and a detector 84 that is operable to detect light 16 that is emitted from a scintillator 15. As print head is scanned over scintillator elements 15 (elements 15-1, 15-2 and 15-3 are shown in FIG. 8) the amount of light 16 detected from each scintillator element is detected (see FIG. 8A).

Output 85 of light 16 from scintillator elements 15 that is stimulated by applying ultraviolet light or other radiation can be used as a feedback mechanism which may be applied during the process of fabricating apparatus 10 to: fix defects in apparatus 10 and/or to cause individual scintillator elements 15 to have variable responses (i.e. emit variable amounts of light 16 in response to a specified radiation intensity) as desired; and/or to provide element-to-element sensitivity corrections.

An amount of scintillator material to be added to complete fabrication of the scintillator element may be based on the monitored intensity. For example: if the monitored intensity is lower than expected (e.g. lower than a “low threshold” 86—the low threshold may be different for different ones of scintillator elements 15) then an amount of scintillator material to be added to complete the scintillator element may be increased to compensate for the lower than expected light output of the partially completed scintillator element 15; if the monitored intensity is higher than expected (e.g. greater than a “high threshold”—the high threshold may be different for different ones of scintillator elements 15) then an amount of scintillator material to be added to complete the scintillator element may be reduced to compensate for the higher than expected light output of the partially completed scintillator element 15.

In some embodiments, a scintillator element is increased in volume by adding successive layers of scintillator material until the feedback indicates that a desired response (i.e. a desired output of light 16) results when the scintillator element 15 is irradiated (e.g. by ultraviolet light).

In some embodiments, a radiation source (e.g. UV light source) is applied during manufacturing of an apparatus 10 to measure relative sensitivities of different scintillator elements 15 (which may have different volumes, sizes etc. This information may be compared to specifications for or estimates of the relative sensitivities of the scintillator elements 15. Estimates of the relative sensitivities of scintillator elements 15 that may be based on a solid model of apparatus 10. Deviations from the specifications or estimates may indicate problems with the apparatus 10 that is being fabricated.

### Example Fabrication Details

The following description provides features that may be included in making apparatus 10.

In experiments that have been conducted to validate the feasibility of the present technology, fused deposition modeling (FDM) was used to create arrays of scintillator elements.

A high-resolution (scintillating element size 3×3×3 mm³) planar scintillator array was fabricated using a BCN3D Epsilon W27 3D printer.

The array was composed of polylactic acid (PLA) filament and BCF-10 plastic scintillator material filament. These filaments had diameter of 3 mm. The array's response was initially characterized using a 20×20 cm² 6 MV photon field with a source-to-surface (SSD) distance of 100 cm and the beam incident on the top of the array. The light signals emitted under irradiation were imaged using 200 ms exposures from a sCMOS camera positioned at the foot of the treatment couch (210 cm from the array). The collected images were then processed using a purpose-built software to correct known optical artefacts and determine the light output for each scintillating element.

In these experiments the scintillator elements were of size 3×3×3 mm³ and were printed using a BCN3D Epsilon W27 3D printer. This printer is an example of an independent dual extruder (IDEX) 3D printer. IDEX printers have independently moving extruders. While not strictly required, the IDEX design offers two distinct advantages over other currently popular designs for dual material 3D printers which move two extruders together. These advantages are decreased material cross contamination and reduced printer head mass. Material cross-contamination can result in occluded scintillators with poor light output. Reduced printer head mass is a factor which tends to facilitate improved dimensional accuracy of printed structures.

Scintillator elements were printed using filaments of BCF-10 plastic scintillator material (Saint Gobain Crystals, Ohio, USA). The rest of the body of the arrays was printed using polylactic acid (PLA) filament. In-process corrections to adjust performance of individual scintillator elements were not performed.

Printing parameters were optimized for the above materials to achieve consistent scintillator array print quality. Scintillator arrays were printed using a nozzle size of 0.4 mm, layer thickness of 0.1 mm, 100% infill density and a build plate temperature of 65° C. PLA was printed using a print temperature of 195° C., line-based infill pattern and a print speed of 35 mm/s with retractions enabled.

Best results for printing the scintillator material were achieved by avoiding printing the scintillator elements too quickly and using an infill pattern in which the number of large changes in direction is reduced. The scintillator material was printed using a print temperature of 210° C., a print speed of 10 mm/s, concentric printing of infill and retractions disabled. These parameters were found to reduce jamming, significant warping, and poor adhesion of the scintillator material filament during the printing process.

The array's response to radiation was characterized using a 20×20 cm² 6 MV photon field with a source-to-surface distance of 100 cm and the beam incident on the top of the array. The light signals emitted under irradiation were imaged using 200 ms exposures from a 16-bit pco.panda 4.2 sCMOS camera (PCO Photonics Ltd., Ontario, Canada) positioned at the foot of the treatment couch (210 cm from the array). The camera was equipped with a 50 mm F/1.8 manual focus lens and aligned perpendicular to the array surface. The image sensor of the camera used in this experiment has a pixel size of 6.5×6.5 μm² and a total of 2048×2048 pixels.

The collected images were then processed using purpose-built software to correct various optical artefacts and determine the light output for each scintillator element 15. Optical artefacts can include both sensor based artefacts and lens-based artefacts. Such artefacts can include:
sensor noise,
background light contamination,
stray radiation,
vignetting, and
lens distortion.
   Methods for correcting for the effects of these artefacts are known in the art (see, e.g. Robertson D, Hui C, Archambault L, Mohan R, Beddar S. Optical artefact characterization and correction in volumetric scintillation dosimetry. Phys Med Biol. 2014; 59 (1): 23-42. doi: 10.1088/0031-9155/59/1/23; Archambault L, Briere™, Beddar S. Transient noise characterization and filtration in CCD cameras exposed to stray radiation from a medical linear accelerator. Med Phys. 2008; 35 (10): 4342-4351. doi: 10.1118/1.2975147; and Jennings M W, Rutten T P, Ottaway D J. Evaluation of the signal quality of an inexpensive CMOS camera towards imaging a high-resolution plastic scintillation detector array. Radiat Meas. 2017; 104:22-31.
   doi: 10.1016/j.radmeas.2017.07.004) and are therefore not discussed here in detail. Embodiments of the present technology may include processing that corrects for the effects of all or some of these artefacts (in any combination) using the approaches described herein or other approaches that are known in the art or may be developed in the future. FIG. 5 shows a non-limiting example sequence of operations that may be used to correct artefacts.

Initially, dark images (no light, no radiation) and background images (light with no radiation) were collected prior to irradiation. Median dark and background images were subtracted from each of the raw images.

A vignetting correction was applied by dividing the processed images by a flat-field image. This correction accounts for variation in pixel-to-pixel sensitivity and the brightness reduction which occurs in pixels near the periphery of each image due to occlusion by the camera aperture. The flat-field image is acquired by imaging a uniformly illuminated surface such as a flat-screen LCD computer monitor diffused by a white sheet of paper.

Following vignetting correction, lens distortion effects resulting from the optical design of the 50 mm camera lens were corrected for using the Camera Calibration function of the MATLAB™ Computer Vision Toolbox. This correction accounts for the slight deformation of straight lines that occurs near the periphery of the image. The calibration is performed using multiple images of a planar calibration pattern (checkerboard) with known dimensions. By acquiring images at different distances, angles, and positions relative to the optical axis of the camera the resulting distortions can be modeled by comparing the calibration pattern dimensions in each image to its known dimensions (see e.g., Zhang Z. A flexible new technique for camera calibration. IEEE Trans Pattern Anal Mach Intell. 2000; 22 (11): 1330-1334. doi: 10.1109/34.888718 and Heikkila J, Silven O. A four-step camera calibration procedure with implicit image correction. In: Proceedings of IEEE Computer Society Conference on Computer Vision and Pattern Recognition; 1997:1106-1112. doi: 10.1109/CVPR.1997.609468).

To remove stray radiation, a rolling temporal median filter may be applied. Temporal median filtering is the preferred method for stray radiation removal as it guarantees that pixel values falling far outside the norm (in this case, transient noise from stray radiation) will be removed from the final processed images. In this experimental embodiment the temporal median filter was implemented by, beginning with a first image, taking five consecutive images (1 s of data in this example) and computing a median image. The filtering then increments to the second image, performs this operation again, and continues iteratively until all the collected images have been processed. The median images are the output of the temporal median filtering In some embodiments spatial median filtering or temporal-spatial median filtering is applied to obtain median images.

Light outputs from the scintillator elements were corrected for element sensitivity and calibrated to dose using Monte Carlo simulations of the array and irradiation geometry based on the digital 3D print model that was used to create the array. To assess the accuracy of the array calibration both a 3D beam and a clinical VMAT plan were delivered. Dose measurements using the calibrated array were then compared to dose measurements made using EBT3 GAFChromic film and OSLD dosimetry, as well as Monte Carlo simulations and TPS calculations.

Assessment of the responses of 3D printed scintillators across each row of the array demonstrated a non-uniform response with an average percent deviation from the mean of 2.1%±2.8%. Array dose measurements performed following calibration indicate difficulty in differentiating the scintillator response from ambient background light contamination at low doses (<20-25 cGy) and dose rates (≤100 MU/min). However, when analysis was restricted to exclude dose values less than 10% of the Monte Carlo simulated maximum dose the average absolute percent dose difference between Monte Carlo simulation and array measurement was 5.3%±4.8% for the fixed beam delivery and 5.4%±5.2% for the VMAT delivery.

Advantages that may be provided by embodiments of the present technology include the ability to provide:
accessible, routine, and real-time in-vivo dosimetry for patients undergoing radiotherapy using patient specific detectors;
accessible personalized dose evaluation during RT treatments;
patient specific device fabrication and detector arrangement;
Incorporation into patient support devices;
combined dose and position verification;
calibration without the need for ionizing radiation′

### Variations

The technology described herein may be varied in a large number of ways. For example, an apparatus 10 may be made flexible so that the apparatus 10 relies at least in part on contact with the anatomy of a patient to maintain its shape. Such embodiments may include arrays of scintillator elements that are configured based on patient specific radiation treatment planning data. Such embodiments preferably include indicia or other means for repeatably placing the flexible apparatus 10 to cover a desired portion of the anatomy of a patient P for which the apparatus was designed.

Where a component (e.g. a software module, processor, assembly, device, circuit, etc.) is referred to herein, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

### INTERPRETATION OF TERMS

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms. These terms (“a”, “an”, and “the”) mean one or more unless stated otherwise;
“and/or” is used to indicate one or both stated cases may occur, for example A and/or B includes both (A and B) and (A or B);
“approximately” when applied to a numerical value means the numerical value±10%;
where a feature is described as being “optional” or “optionally” present or described as being present “in some embodiments” it is intended that the present disclosure encompasses embodiments where that feature is present and other embodiments where that feature is not necessarily present and other embodiments where that feature is excluded. Further, where any combination of features is described in this application this statement is intended to serve as antecedent basis for the use of exclusive terminology such as “solely,” “only” and the like in relation to the combination of features as well as the use of “negative” limitation(s)” to exclude the presence of other features; and
“first” and “second” are used for descriptive purposes and cannot be understood as indicating or implying relative importance or indicating the number of indicated technical features.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a range for a value is stated, the stated range includes all sub-ranges of the range. It is intended that the statement of a range supports the value being at an endpoint of the range as well as at any intervening value to the tenth of the unit of the lower limit of the range, as well as any subrange or sets of sub ranges of the range unless the context clearly dictates otherwise or any portion(s) of the stated range is specifically excluded. Where the stated range includes one or both endpoints of the range, ranges excluding either or both of those included endpoints are also included in the invention.

Certain numerical values described herein are preceded by “about”. In this context, “about” provides literal support for the exact numerical value that it precedes, the exact numerical value±5%, as well as all other numerical values that are near to or approximately equal to that numerical value. Unless otherwise indicated a particular numerical value is included in “about” a specifically recited numerical value where the particular numerical value provides the substantial equivalent of the specifically recited numerical value in the context in which the specifically recited numerical value is presented. For example, a statement that something has the numerical value of “about 10” is to be interpreted as: the set of statements:
in some embodiments the numerical value is 10;
in some embodiments the numerical value is in the range of 9.5 to 10.5;
   and if from the context the person of ordinary skill in the art would understand that values within a certain range are substantially equivalent to 10 because the values with the range would be understood to provide substantially the same result as the value 10 then “about 10” also includes:
in some embodiments the numerical value is in the range of C to D where C and D are respectively lower and upper endpoints of the range that encompasses all of those values that provide a substantial equivalent to the value 10.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

As will be apparent to those of skill in the art upon reading this disclosure, each of the individual embodiments described and illustrated herein has discrete components and features which may be readily separated from or combined with the features of any other described embodiment(s) without departing from the scope of the present invention.

Any aspects described above in reference to apparatus may also apply to methods and vice versa.

Any recited method can be carried out in the order of events recited or in any other order which is logically possible. For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, simultaneously or at different times.

Various features are described herein as being present in “some embodiments”. Such features are not mandatory and may not be present in all embodiments. Embodiments of the invention may include zero, any one or any combination of two or more of such features. All possible combinations of such features are contemplated by this disclosure even where such features are shown in different drawings and/or described in different sections or paragraphs. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible). This is the case even if features A and B are illustrated in different drawings and/or mentioned in different paragraphs, sections or sentences.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. Radiation dosimetry apparatus comprising:
a body having a first surface formed to conform with contours of a portion of a patient's anatomy to which radiation is to be delivered according to a patient-specific radiation treatment plan, the radiation treatment plan specifying radiation dose as a function of location on the portion of the patient's anatomy;
a plurality of scintillator elements in the body and visible on a second surface of the body opposed to the first surface, the scintillator elements comprising a material that emits scintillation light in response to the radiation specified by the radiation treatment plan.

2. The radiation dosimetry apparatus according to claim 1 wherein:
the radiation treatment plan includes at least one higher dose area within which the specified radiation dose has a magnitude at least equal to a first value, at least one lower dose area within which the specified radiation dose has a magnitude that is less than or equal to a second value that is smaller than the first value; and a transition region located between the higher dose area and the lower dose area within which the specified radiation dose has values between the first value and the second value;
the scintillator elements include at least some scintillator elements in the high dose region and at least some scintillator elements in the transition region.

3.-4. (canceled)

5. The radiation dosimetry apparatus according to claim 2 wherein a density of the scintillator elements in the high dose region is greater than a density of the scintillator elements in the low dose region.

6. The radiation dosimetry apparatus according to claim 2 wherein a density of the scintillator elements in the transition region is greater than a density of the scintillator elements in the high dose region.

7. The radiation dosimetry apparatus according to claim 2 wherein the low dose region comprises a OAR region corresponding to an organ at risk (OAR) and the scintillator elements in the OAR region have higher sensitivity to radiation than the scintillator elements in the high dose region.

8. (canceled)

9. The radiation dosimetry apparatus according to claim 7 wherein the scintillation light emitted by the scintillator elements in the OAR region has a spectral composition different from the spectral composition of scintillation light emitted by the scintillator elements located outside of the OAR region.

10. The radiation dosimetry apparatus according to claim 2 wherein at least in a neighbourhood of a boundary between the high dose region and the transition region the scintillator elements are elongated and oriented transversely to a line extending through the transition region from the high dose region to the low dose region.

11. The radiation dosimetry apparatus according to claim 2 wherein at least in a neighbourhood of a boundary between the high dose region and the transition region the scintillator elements are smaller than the scintillator elements in the low dose region.

12. (canceled)

13. The radiation dosimetry apparatus according to claim 2 wherein at least in a neighbourhood of a boundary between the high dose region and the transition region the scintillator elements are staggered radially relative to the location of the boundary.

14. The radiation dosimetry apparatus according to claim 1 wherein the radiation dosimetry apparatus is designed for use in a system which includes a camera positioned to image the radiation dosimetry apparatus from a camera direction and a plurality of the scintillator elements are configured to direct the scintillator light from the scintillator element preferentially in the camera direction FF wherein ends of the plurality of scintillator elements facing away from the first surface are shaped to preferentially direct the scintillation light in the camera direction.

15.-22. (canceled)

23. The radiation dosimetry apparatus according to claim 1 wherein the body is stiff and holds the configuration of the first surface.

24. (canceled)

25. The radiation dosimetry apparatus according to claim 24 wherein the body comprises a material that is optically clear at least at a wavelength of the scintillation light.

26. The radiation dosimetry apparatus according to claim 1 wherein the body comprises at least one transparent window and indicia in the window that may be aligned with a fiducial marking on the patient that is visible through the window when the radiation dosimetry apparatus is engaged against the portion of the patient's anatomy.

27.-28. (canceled)

29. The radiation dosimetry apparatus according to claim 1 wherein the body includes a light reflective layer between the scintillator elements and the first surface.

30.-31. (canceled)

32. The radiation dosimetry apparatus according to claim 1 wherein the scintillator elements are surrounded or partially surrounded by a material that has an index of refraction significantly lower than an index of refraction of the material of the scintillator elements such that the scintillation light is guided out of the scintillator elements at least in part by total internal reflection.

33.-38. (canceled)

39. A system for radiation treatment, the system comprising the radiation dosimetry apparatus according to any of the preceding claims combined with a radiation delivery system, at least one camera arranged to image the radiation dosimetry apparatus and a control apparatus connected to receive images from the at least one camera;
wherein the control apparatus is configured to:
process the images to obtain measures of the scintillation light emitted by the scintillator elements of the radiation dosimetry apparatus;
determine corresponding measured radiation intensities of radiation from the radiation delivery system at the scintillation elements; compare the measured radiation intensities to planned radiation intensities provided by the radiation treatment plan; and
take an action if the measured radiation intensities deviate significantly from the planned radiation intensities.

40.-42. (canceled)

43. The system according to claim 39 wherein the control apparatus stores separate calibration information for each of the scintillator elements, the calibration information specifying a relationship between the measure of the scintillation light emitted by the corresponding scintillator element and the measured radiation intensity at the location of the corresponding scintillator element, wherein the calibration information compensates for the geometrical relationship of the camera and the radiation dosimetry apparatus.

44. (canceled)

45. The system according to claim 39 wherein the at least one camera comprises first and second cameras and the control apparatus is configured to perform stereo processing using images from the first and second cameras wherein:
the stereo processing comprises determining a pose of the radiation dosimetry apparatus relative to the first and second cameras;
the control apparatus is configured to monitoring the pose to detect motion of the radiation dosimetry apparatus; and
the control apparatus is configured to take an action in response to detecting motion of the radiation dosimetry apparatus.

46.-47. (canceled)

48. The system according to claim 39 wherein the control apparatus is configured to generate a record of delivered radiation dose as a function of time for each of the scintillator elements.

49.-50. (canceled)

51. The system according to claim 39 wherein the radiation delivery system is operable to emit radiation pulses and operation of shutters of the one or more cameras is synchronized to the pulses of radiation emitted by the radiation delivery system.

52.-72. (canceled)
